(19) United States
(12) Patent Application Publication
(10) Pub. No.: US 2004/0210117 A1
(43) Pub. Date: Oct. 21, 2004

(54) Behavior control support apparatus and method

(75) Inventors: Ken Ueno, Kanagawa-ken (JP); Shigeaki Sakurai, Tokyo (JP)

Correspondence Address: Finnegan, Henderson, Farabow,, Garrett & Dunner, L.L.P., 1300 I Street, N.W., Washington, DC, 20005-3315, US

(73) Assignee: KABUSHIKI KAISHA TOSHIBA

(21) Appl. No.: 10/808,562

(22) Filed: Mar. 25, 2004

(30) Foreign Application Priority Data
Apr. 16, 2003 (JP) P2003-111670

(51) Int. Cl. A61B 5/00

(52) U.S. Cl. 600/300; 128/920; 705/2

(57) ABSTRACT

An integrated behavior database generation unit generates an integrated behavior database. The integrated behavior database correspondingly stores biomedical information and behavior relational information of a user. The biomedical information is detected by a sensor associated with the user's body. A behavior rule generation unit generates a behavior rule of the user by referring to the integrated behavior database. A message generation unit generates a message to urge the user to do an exercise by referring to the behavior rule. A message notice unit notifies the user of the message.

### CROSS-REFERENCE TO RELATED APPLICATIONS

This application is based upon and claims the benefit of priority from prior Japanese Patent Application P2003-111670, filed on Apr. 16, 2003; the entire contents of which are incorporated herein by reference.

### FIELD OF THE INVENTION

The present invention relates to a behavior control support apparatus and method for obtaining various status of a user by a device attachable to the user's body and for supporting the user's behavior using the various status.

### BACKGROUND OF THE. INVENTION

An apparatus for supporting a user's behavior is disclosed in Japanese Patent Disclosure (Kokai) PH09-103413 (For example, paragraph numbers [0060]˜[0063], FIGS. 15˜17). In this apparatus, personal daily biomedical information such as calorie consumption quantity, temperature, base temperature, blood pressure, heart beat, stress degree, blood sugar value, urine sugar value, urine protein, sleep degree, body fat ratio, or body measurements of the user are obtained. A personal physiological biorhythm is determined from the obtained physiological information. By informing the personal physiological biorhythm with the obtained physiological information to the user, the user can recognize a cause of quality of physiological status. This apparatus can urge the user to do an exercise at a suitable time. For example, a diet can be indicated on a day when the user can easily lose weight.

However, in this apparatus, a daily behavior rule of each user is not taken into consideration. Accordingly, it is difficult to explicitly point out a reason of exercise promotion to the user. Furthermore, it is difficult to pliably generate an exercise schedule in daily life as time level. Briefly, customization of exercise is due to personal intention.

On the other hand, another apparatus for supporting a user's behavior is disclosed in Japanese Patent Disclosure (Kokai) PH10-118052 (For example, paragraph numbers [0040]˜[0051], FIG. 5). In this apparatus, in addition to exercise quantity to consume in usual life, exercise quantity to maintain health is guided to the user. The user's health control can be realized by just sufficient enough exercise. Briefly, the exercise quantity including calorie consumed in daily life except for supports can be calculated, and the exercise quantity necessary for accomplishing the user's purpose is indicated by considering personal information such as age, gender, and body type.

However, in this apparatus, the exercise quantity to accomplish the target value is only presented based on registered target number of heart beats. Customization of the exercise is due to personal intention. Briefly, in order to raise the user's motivation and customize the exercise, a suitable advice is necessary to be delicately presented and a timing to present the advice is important. By generating a daily behavior rule of the user, it is effective to utilize the daily behavior rule as one element. However, such apparatus is unknown.

As a method for generating a personal behavior rule, following method is known.

GSP (R. Srikant, R. Agrawal., “Mining Sequential Patterns: Generalizations and Performance Improvements”, Proc. 5th Int. Conf. Extending Database Technology, EDBT, pp.3-17,1996) . . . (1)

PrefixSpan (J. Pei, J. Han, B. Mortazavi-Asl, H. Pinto, Q. Chen, U. Dayal, Mei-Chun Hsu, “PrefixSpan: Mining Sequential Patterns Efficiently by Prefix-Projected Pattern Growth”, Proc. of International Conference of Data Engineering (ICDE2001), pp. 215-224, 2001)

In these methods, time is segmented by a fixed length, and a personal behavior rule is generated in each period.

As mentioned-above, in the prior art, increase of the user's exercise quantity is due to the user's intention. Especially, in order to customize the exercise, it is desired to lighten the user's burden.

### SUMMARY OF THE INVENTION

The present invention is directed to a behavior control support apparatus and a method able to naturally increase exercise quantity for the user in daily life.

According to an aspect of the present invention, there is provided an apparatus for supporting a user's behavior, comprising: an integrated behavior database generation unit configured to generate an integrated behavior database correspondingly storing a biomedical information and a behavior relational information of a user, the biomedical information being detected by a sensor associated with the user's body; a behavior rule generation unit configured to generate a behavior rule of the user by referring to the integrated behavior database; a message generation unit configured to generate a message to urge the user to do an exercise by referring to the behavior rule; and a message notice unit configured to notify the user of the message.

According to another aspect of the present invention, there is also provided a method for supporting a user's behavior, comprising: generating an integrated behavior database correspondingly storing a biomedical information and a behavior relational information of the user, the biomedical information being detected by a sensor associated with the user's body; generating a behavior rule of the user by referring to the integrated behavior database; generating a message to urge the user to do an exercise by referring to the behavior rule; and notifying the user of the message.

According to still another aspect of the present invention, there is also provided a computer program product, comprising: a computer readable program code embodied in said product for causing a computer to support a user's behavior, said computer readable program code comprising: a first program code to generate an integrated behavior database correspondingly storing a biomedical information and a behavior relational information of the user, the biomedical information being detected by a sensor associated with the user's body; a second program code to generate a behavior rule of the user by referring to the integrated behavior database; a third program code to generate a message to urge the user to do an exercise by referring to the behavior rule; and a fourth program code to notify the user of the message.

### BRIEF DESCRIPTION OF THE DRAWINGS

FIG. 1 is a block diagram of a system including a behavior control support apparatus.

FIG. 2 is a block diagram of a first embodiment of a behavior control support apparatus.

FIG. 3 is a schematic diagram of one example of contents in a personal attribute data set 1.

FIG. 4 is a schematic diagram of one example of contents in a behavior data set 21.

FIG. 5 is a schematic diagram of one example of contents in a feeling data set 22.

FIG. 6 is a schematic diagram of one example of contents in a behavior schedule data set 23.

FIG. 7 is a schematic diagram of one example of contents in a sensor data set 3.

FIG. 8 is a schematic diagram of one example of contents in an integrated behavior data set 7.

FIG. 9 is a schematic diagram of one example of contents in a behavior rule set 8.

FIG. 10 is a schematic diagram of one example of contents in a concept dictionary data set contained in related data set 9.

FIG. 11 is a schematic diagram of one example of contents in a behavior label set contained in related data set 9.

FIG. 12 is a schematic diagram of one example of contents in a calendar weather data set contained in related data set 9.

FIG. 13 is a schematic diagram of one example of contents in a route data set contained in related data set 9.

FIG. 14 is a schematic diagram of one example of contents in a location data set contained in related data set 9.

FIG. 15 is a schematic diagram of one example of contents in a map data set contained in related data set 9.

FIG. 16 is a schematic diagram of one example of contents in a map relational data set contained in related data set 9.

FIG. 17 is a schematic diagram of one example of contents in a behavior advice set 10.

FIG. 18 is a schematic diagram of one example of contents in an exercise constraint condition rule set 12.

FIGS. 19A and 19B are flow charts of processing of the behavior control support apparatus.

FIG. 20 is a schematic diagram of point generation of behavior description processing on a data input interface C4.

FIG. 21 is a schematic diagram of point definition of behavior description processing on the data input interface C4.

FIG. 22 is a schematic diagram of move definition of behavior description processing on the data input interface C4.

FIG. 23 is a schematic diagram of departure of behavior description processing on the data input interface C4.

FIG. 24 is a schematic diagram of arrival of behavior description processing on the data input interface C4.

FIG. 25 is a schematic diagram of behavior record of behavior description processing on the data input interface C4.

FIG. 26 is a schematic diagram of addition of relay point of behavior description processing on the data input interface C4.

FIG. 27 is a schematic diagram of arrival of present place of behavior description processing on the data input interface C4.

FIG. 28 is a schematic diagram of behavior record of behavior description processing on the data input interface C4.

FIG. 29 is a schematic diagram of departure from present place of behavior description processing on the data input interface C4.

FIG. 30 is a schematic diagram of returning home of behavior description processing on the data input interface C4.

FIG. 31 is a schematic diagram of generation of contents of the integrated behavior data set 7.

FIG. 32 is a schematic diagram of reorganization of contents of the behavior schedule data set 23.

FIG. 33 is a schematic diagram of one example of a behavior graph on the data input interface C4.

FIG. 34 is a block diagram of a second embodiment of a behavior control support apparatus.

FIG. 35 is a block diagram of a third embodiment of a behavior control support apparatus.

### DETAILED DESCRIPTION OF THE EMBODIMENTS

Hereinafter, various embodiments of the present invention will be explained by referring to the drawings.

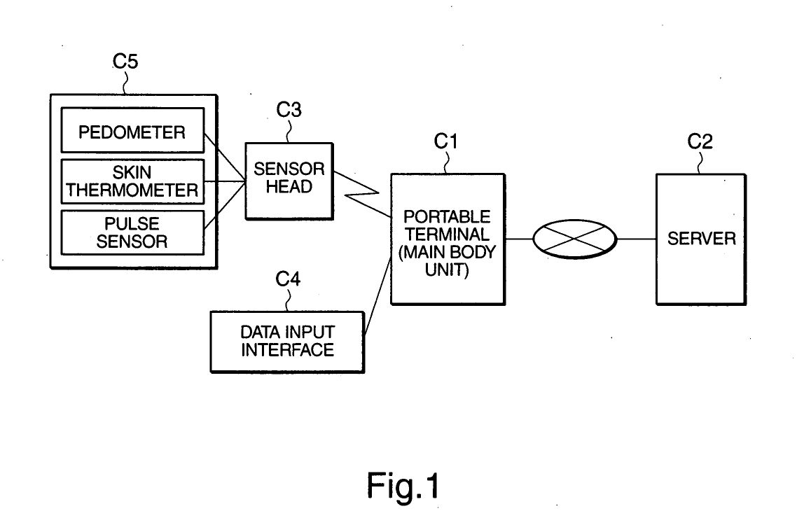

FIG. 1 is a block diagram of a system including a behavior control support apparatus. In this system, a user carries the behavior control support apparatus (Hereafter, a main body unit C1), and a task unprocessed by the main body unit C1 is supplemently processed by a server apparatus C2. The main body unit C1 is realized as a PC (Personal Computer), a PDA (Personal Digital Assistant), a cellular-phone, a PHS, or a wristwatch. It can be a specific device for the behavior control support. The main body unit C1 and the server apparatus C2 are connected through an information communication network such as an Internet, and necessary information is mutually delivered. The interface between the main body unit C1 and the server apparatus C2 may be either wired line or wireless line.

A sensor head C3 is connected to the main body unit C1 through a wired line or wireless line such as Bluetooth (registered trademark). A group of sensors C5 including, for example, a pedometer, a skin thermometer and a pulse sensor, is connected to the sensor head C3. The sensor head C3 collects the user's biomedical information obtained by the group of sensors C5, and transmits it to the main body unit C1. Furthermore, a data input interface C4 is connected to the main body unit C1. The data input interface C4 is realized by, for example, a key board, a tablet, or a speech input interface.

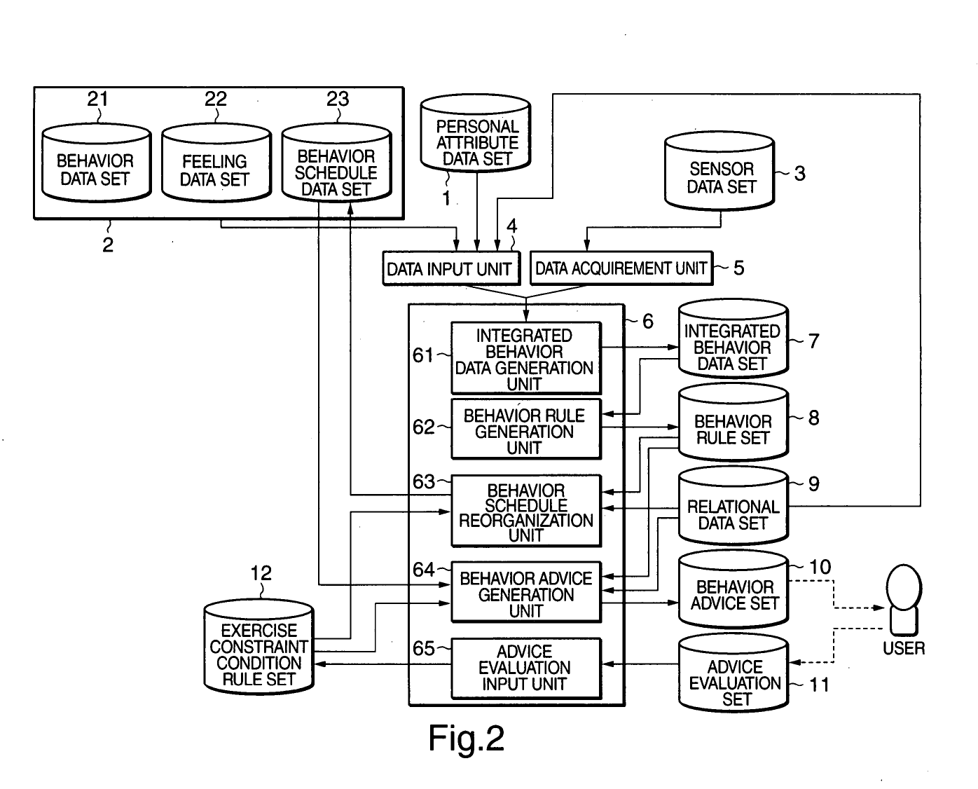

FIG. 2 is a block diagram of the behavior control support apparatus according to the first embodiment. A behavior relational data group 2 stores data related to the user's behavior, and a behavior data processing unit 6 processes various data through a data input unit 4. A data acquirement unit 5 acquires data. Furthermore, this apparatus includes a personal attribute data set 1, a sensor data set 3, an integrated behavior data set 7, a behavior rule set 8, a relational data set 9, a behavior advice set 10, a behavior evaluation set 11, and an exercise constraint condition rule set 12. These data sets are stored in a predetermined database. The behavior relational data group 2 includes a behavior data set 21, a feeling data set 22, and a behavior schedule data set 23. These data sets are also stored in the predetermined database. The behavior data processing unit 6 includes an integrated behavior data generation unit 61, a behavior rule generation unit 62, a behavior schedule reorganization unit 63, a behavior advice generation unit 64, and an advice evaluation input unit 65.

The integrated behavior data generation unit 61 obtains the personal attribute data set 1, the behavior relational data group 2, and the relational data set 9 through the data input unit 4, and obtains the sensor data set 3 through the data acquirement unit 5. The integrated behavior data generation unit 61 relates these data sets in time series, and generates the integrated behavior data set 7.

The behavior rule generation unit 62 generates the user's behavior rule from the integrated behavior data set 7, and generates the behavior rule set 8. In this case, for example, the method for generating the personal behavior rule disclosed in the above-mentioned reference (1) is utilized as an improvement method for this apparatus. The behavior schedule reorganization unit 63 adjusts the user's exercise quantity by referring to the behavior rule set 8, and reorganizes the behavior schedule data set 23 in order to urge an effective exercise. The behavior advice generation unit 64 generates a message to urge the user to do the exercise from the behavior schedule data set 23 reorganized by the behavior schedule reorganization unit 63, the behavior rule set 8, and the relational data set 9. This message is output through a display (not shown in FIG.), for example, a liquid crystal display, of the main body unit C1 to inform the user. The advice evaluation input unit 65 obtains advice evaluation set 11, which contains the user's evaluation for the informed through the data input interface C4. Furthermore, the advice evaluation input unit 65 integrates the user's evaluation and the user's behavior result for the message, and stores the integrated data in the exercise constraint condition rule set 12. The exercise constraint condition rule set 12 is reused as input data by the behavior schedule reorganization unit 63 and the behavior advice generation unit 64.

As used herein, those skilled in the art will understand that the term “unit” is broadly defined as a processing device (such as a server, a computer, a microprocessor, a microcontroller, a specifically programmed logic circuit, an application specific integrated circuit, a discrete circuit, etc.) that provides the described communication and functionally desired. While such a hardware-based implementation is clearly described and contemplated, those skilled in the art will quickly recognize that a “unit” may alternatively be implemented as a software module that works in combination with such a processing device. In addition, one processing device may comprise one or more than one unit. Similarly, “a memory” may refer to one physical memory or several “memories” may be configured on one physical unit.

Depending on the implementation constraints, such a software module or processing device may be used to implement more than one “unit” as disclosed and described herein. Those skilled in the art will be familiar with particular and conventional hardware suitable for use when implementing an embodiment of the present invention with a computer or other processing device. Likewise, those skilled in the art will be familiar with the availability of different kinds of software and programming approaches suitable for implementing one or more “units” as one or more software modules.

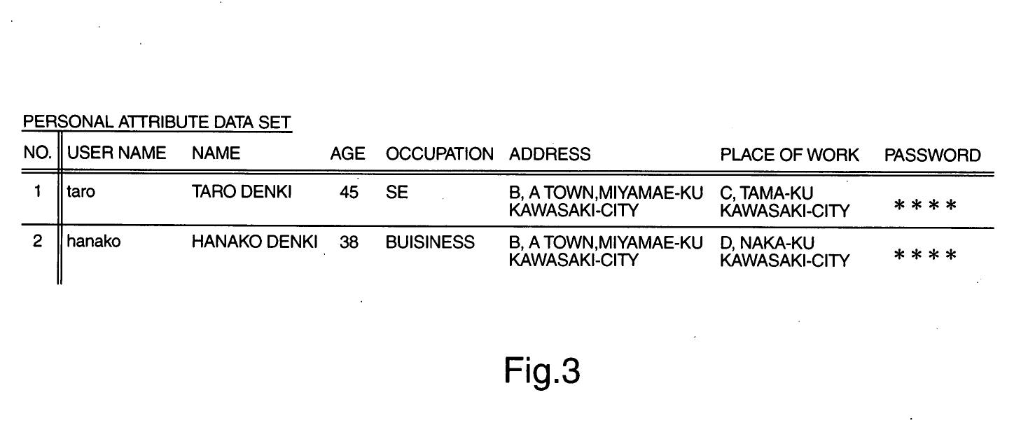

FIG. 3 is a schematic diagram of one example of contents in the personal attribute data set 1. The personal attribute data set 1 is a database in which data such as user name, name, age, gender, occupation, address, place of work, and password are mutually related.

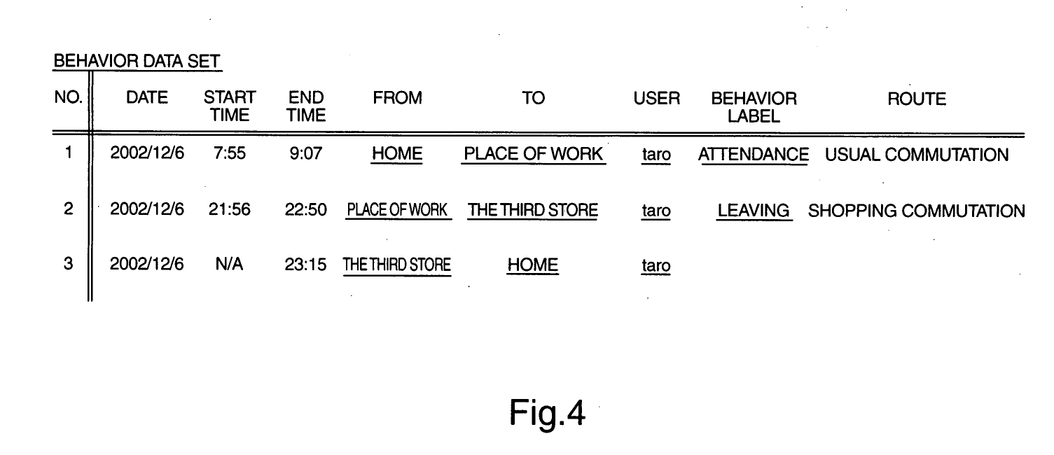

FIG. 4 is a schematic diagram of one example of contents in the behavior data set 21. The behavior data set 21 is a database in which data such as data, start time, end time, present point (FROM), destination point (TO), user name, behavior label, and route where the user traced are mutually related.

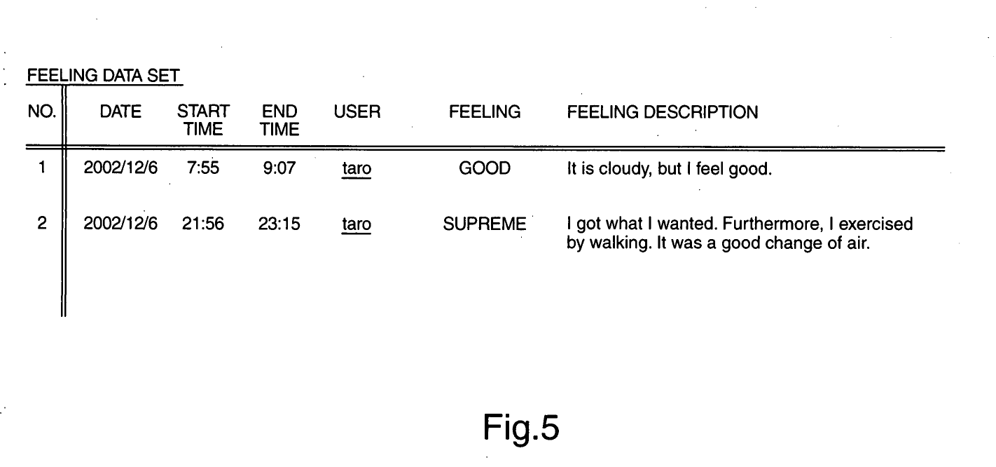

FIG. 5 is a schematic diagram of one example of contents in the feeling data set 22. The feeling data set 22 is a database in which data such as date, start time, end time, user name, feeling, and feeling description input by the user are mutually related.

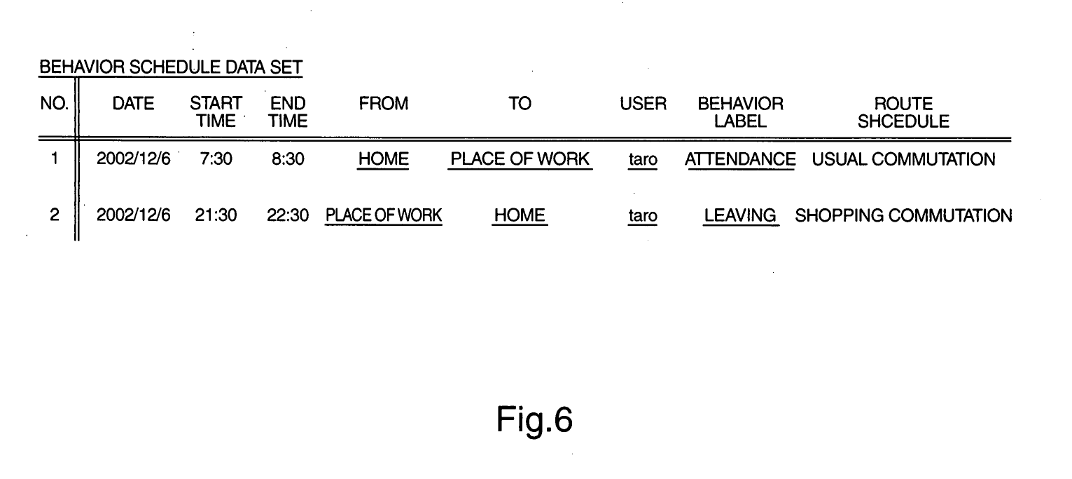

FIG. 6 is a schematic diagram of one example of contents in the behavior schedule data set 23. The behavior schedule data set 23 is previously created based on the user's intention, and is a database in which date, start time, end time, present place (FROM), destination point (TO), user name, behavior label, and route schedule where the user will trace are mutually related.

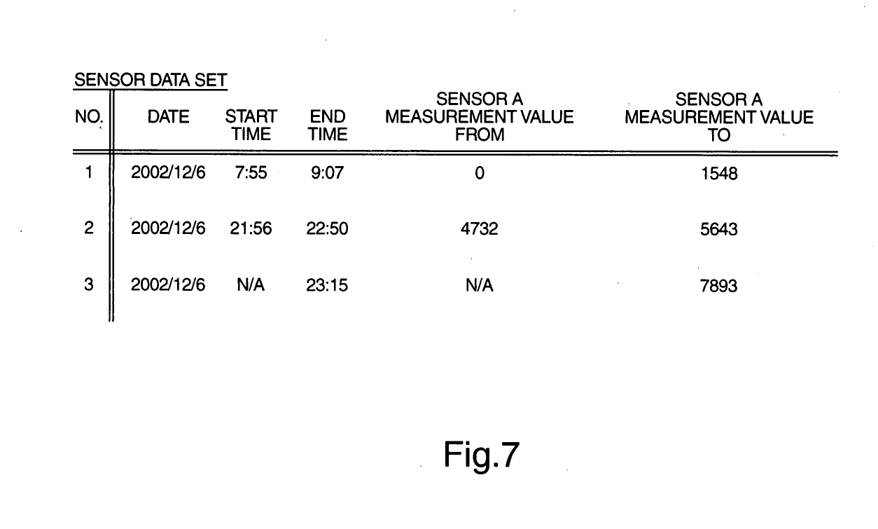

FIG. 7 is a schematic diagram of one example of contents in the sensor data set 3. The sensor data set 3 is a database in which data such as date, start time and end time of biomedical information from sensors C5, sensor measurement value (FROM) at a move source, sensor measurement value (TO) at a move destination are mutually related.

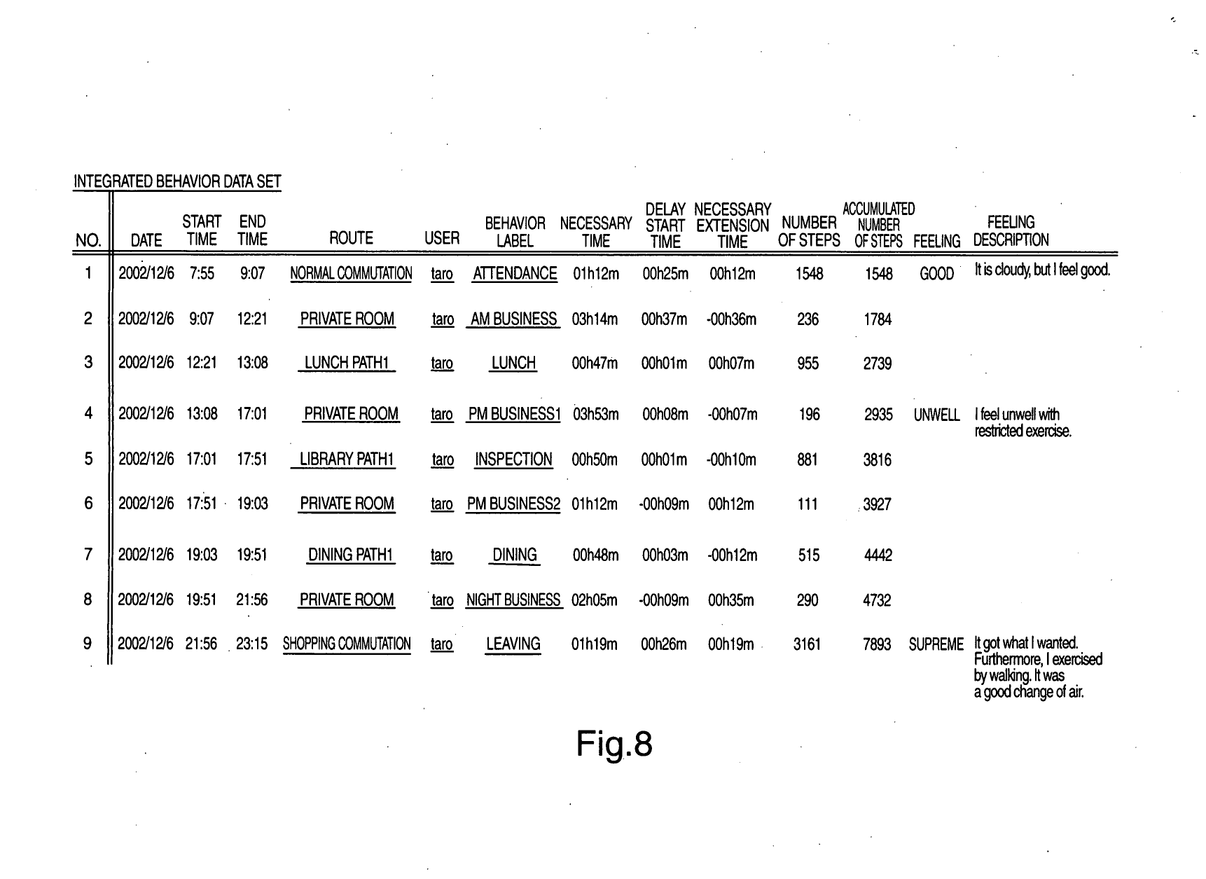

FIG. 8 is a schematic diagram of one example of contents in the integrated behavior data set 7. The integrated behavior data set 7 is a database in which date, start time, end time, route, user name, behavior label, necessary time, delay start time, necessary extension time, number of steps, accumulated number of steps, feeling, and feeling description are mutually related. The integrated behavior data set 7 is generated by mutually relating the recorded contents of the personal attribute data set 1, the behavior data set 21, the feeling data set 22, the behavior schedule data set 23, and the sensor data set 3.

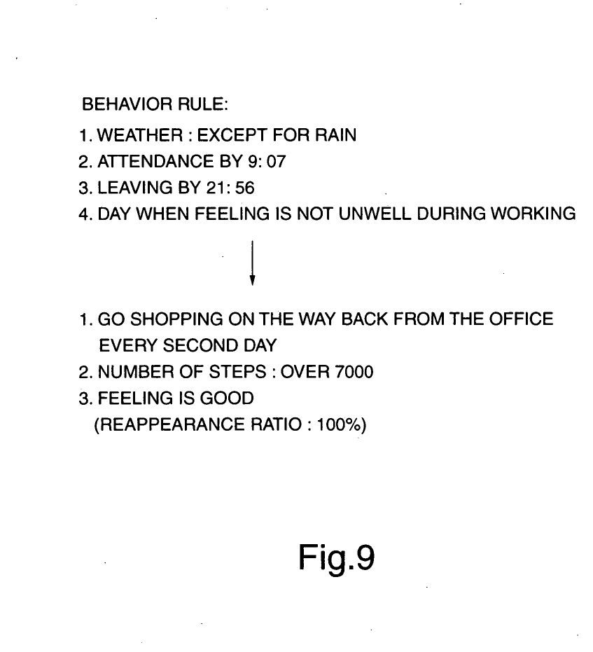

FIG. 9 is a schematic diagram of one example of contents in the behavior rule set 8. The behavior rule set 8 is a database of the user's behavior rule generated from the integrated behavior data set 7 by the behavior rule generation unit 62. For example, on a day when the user's feeling is good during usual business, the user's tendency that the user goes shopping every second day on the way back from the office (except for rainy days) is shown. Furthermore, increase of the number of steps by going shopping is shown. However, if efficiency of business is bad, the increase of the number of steps is meaningless. Accordingly, the user enters the efficiency of business into the feeling description.

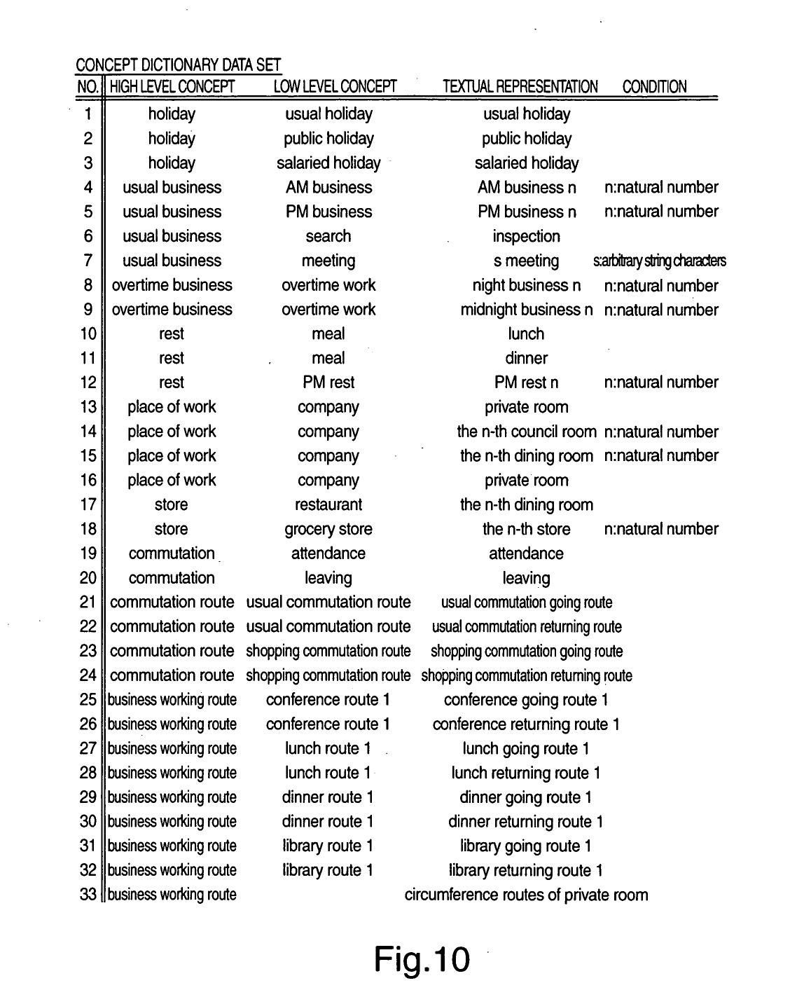

FIG. 10 is a schematic diagram of one example of contents in a concept dictionary data set as a part of the relational data set 9. The concept dictionary data set includes four items such as high level concept, low level concept, textual representation, and condition. For example, the high level concept “rest” has two low level concepts “meal” and “PM rest”. Furthermore, the low level concept “meal” has two textual representations “lunch” and “dinner”. By using this concept dictionary data set, various concepts are determined to represent as which concept level. Accordingly, division or arrangement of numerical data accompanied with text information and the behavior label is operated.

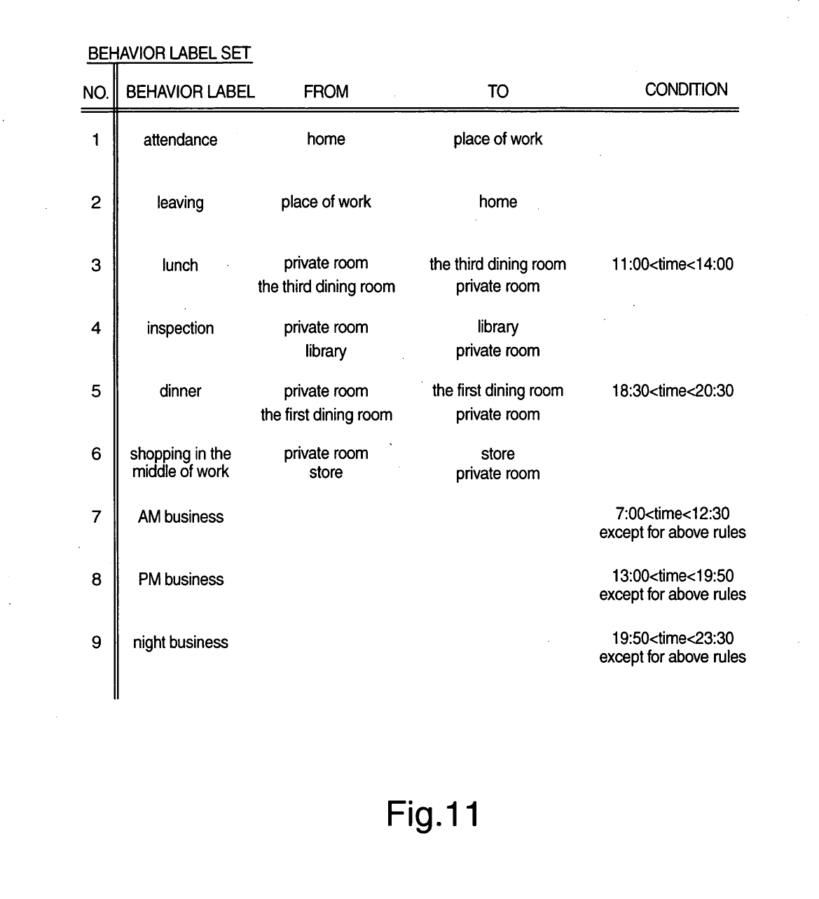

FIG. 11 is a schematic diagram of one example of contents in a behavior label set as a part of the relational data set 9. The behavior label set includes a behavior label, a departure point (FROM), an arrival point (TO), and a condition. A name of the behavior is fixed by determining the departure point and the arrival point. By referring to the behavior label set using the departure point (FROM) and the arrival point (TO) as keywords, the behavior label is specified. If an additional condition exists in each behavior label, it is entered into a condition column.

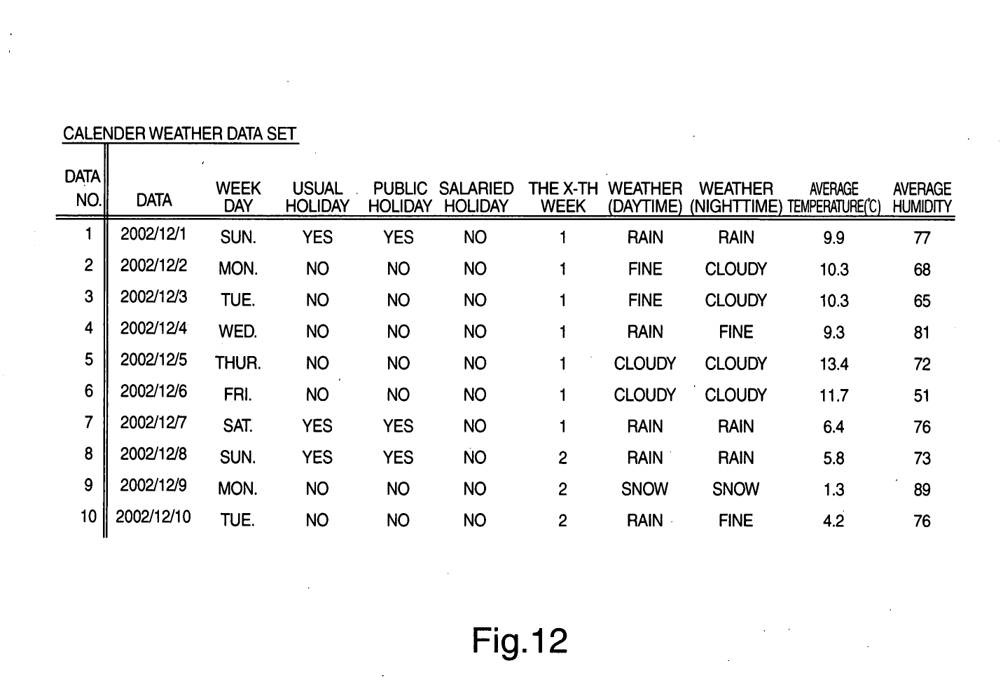

FIG. 12 is a schematic diagram of one example of contents in a calendar weather data set as a part of the relational data set 9. The calendar weather data set 9 is a database in which a date, a weekday of the date, whether it is a usual holiday, whether it is a public holiday, whether it is a salaried holiday, which week including the date, weathers of day time and night time, an average of temperature, and an average of humidity are corresponded. These data are collected from another database or recorded as a measurement value by another sensor.

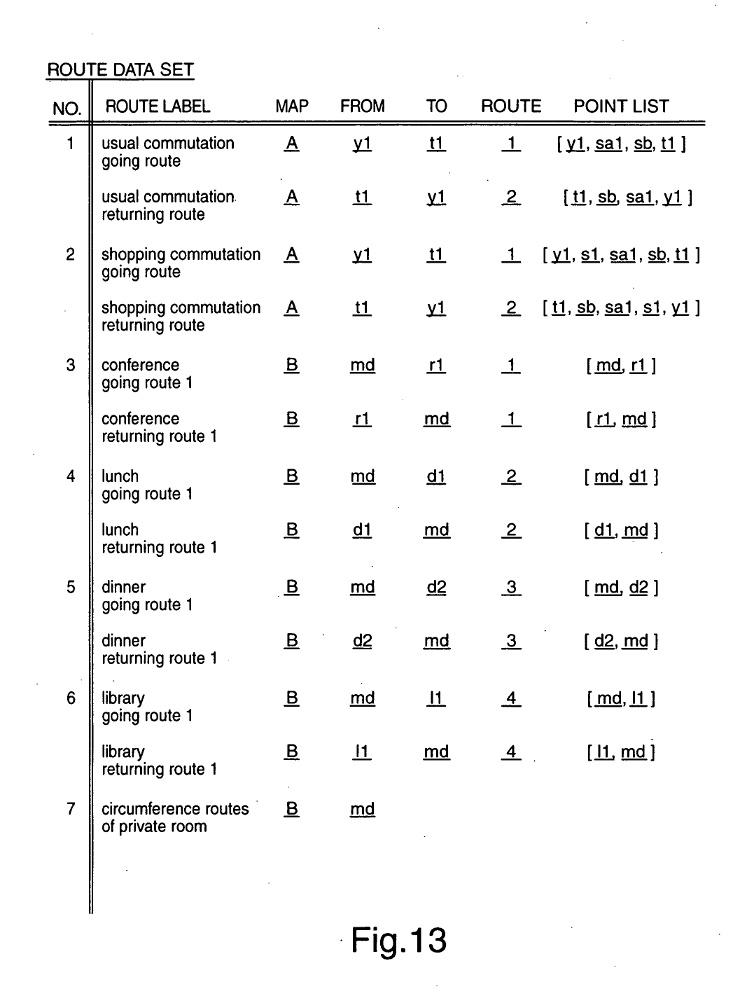

FIG. 13 is a schematic diagram of one example of contents in a route data set as a part of the relational data set 9. The route data set is a database in which a route label, a map, a departure point (FROM), an arrival point (TO), a route, and a point list are corresponded.

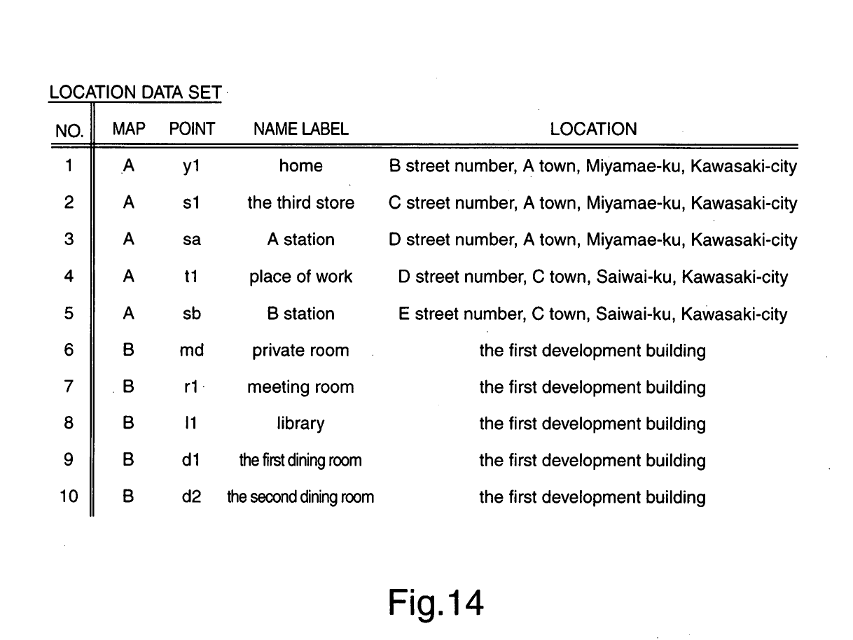

FIG. 14 is a schematic diagram of one example of contents in a location data set as a part of the relational data set 9. The location data set is a database in which a point, a name label, and the location are corresponded with map information recorded as a map data, for example, a bit map format, a vector format, and so on. By using this database, it is recognized that each place represents which point on which map and where the location is.

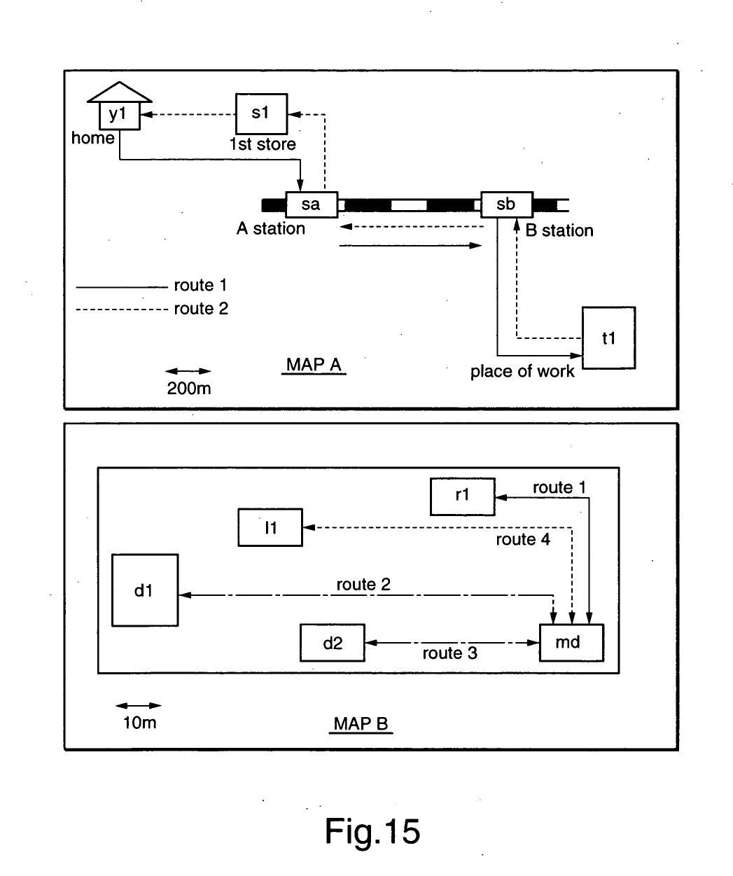

FIG. 15 is a schematic diagram of one example of contents in a map data set as a part of the relational data set 9. The map data set is a database in which a point and a route are corresponded with map information included in the location data set. By referring to this database, relationship among points and routes and information that each point exists on which position of the map data can be known.

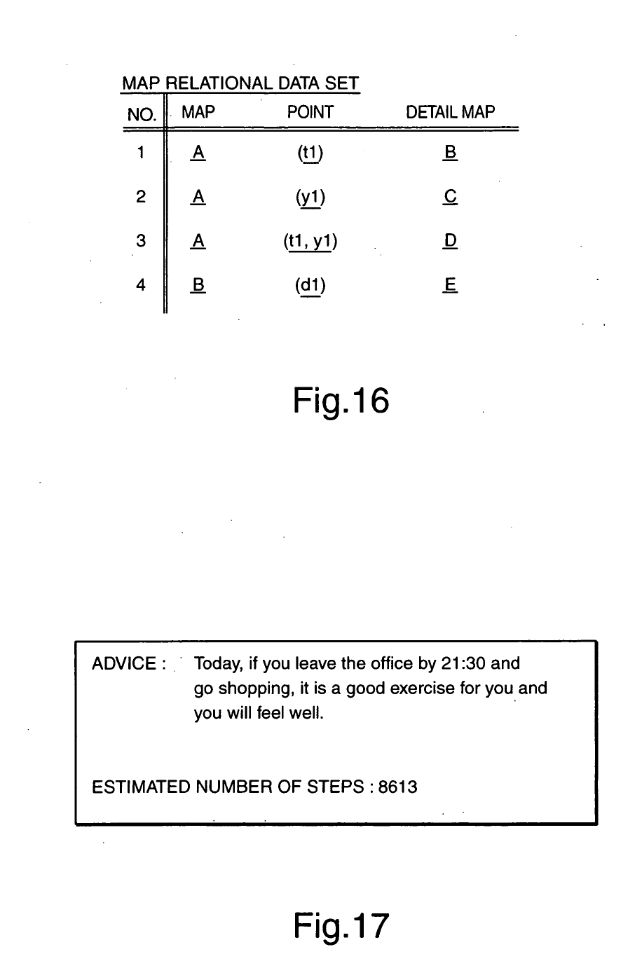

FIG. 16 is a schematic diagram of one example of contents in a map relational data set as a part of the relational data set 9. The map relational data set is a data set representing a relationship between maps. By referring to this data, a relationship where data corresponding to detail map of a certain part of a certain map exist can be known. Above-mentioned relational data set 9 is suitably used at each phase of processing steps explained later.

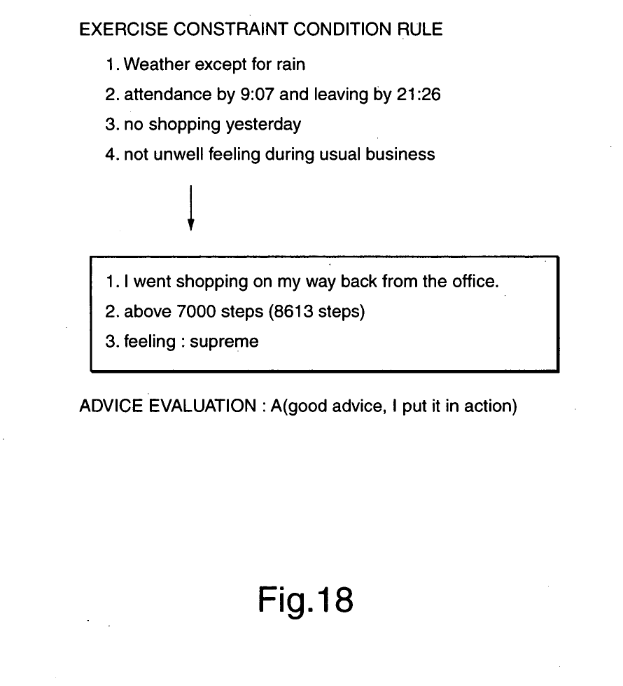

FIG. 17 is a schematic diagram of one example of contents in the behavior advice set 10. The behavior advice set 10 is a database in which an advice (message) presented to the user and an estimated number of steps are corresponded. The estimated number of steps is a prediction value calculated from data history of the number of steps for the past behavior based on the behavior schedule data set 23. The prediction value may be calculated as an average processing, a recursive analysis result, or a value of the center of gravity of clustering.

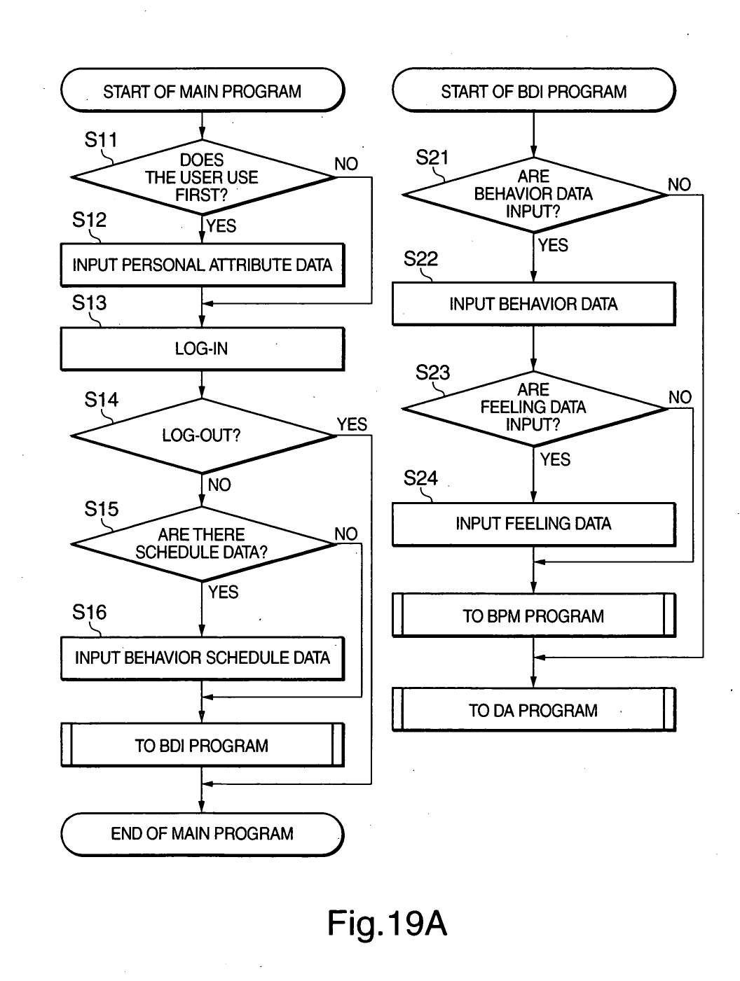

FIG. 18 is a schematic diagram of one example of contents of the exercise constraint condition rule set 12. The exercise constraint condition rule set 12 is a database in which the user's evaluation for the message and the recorded contents of the behavior advice set 10 are corresponded. By referring to this database, the system can generate a message matched with liking and characteristic of each user's behavior.

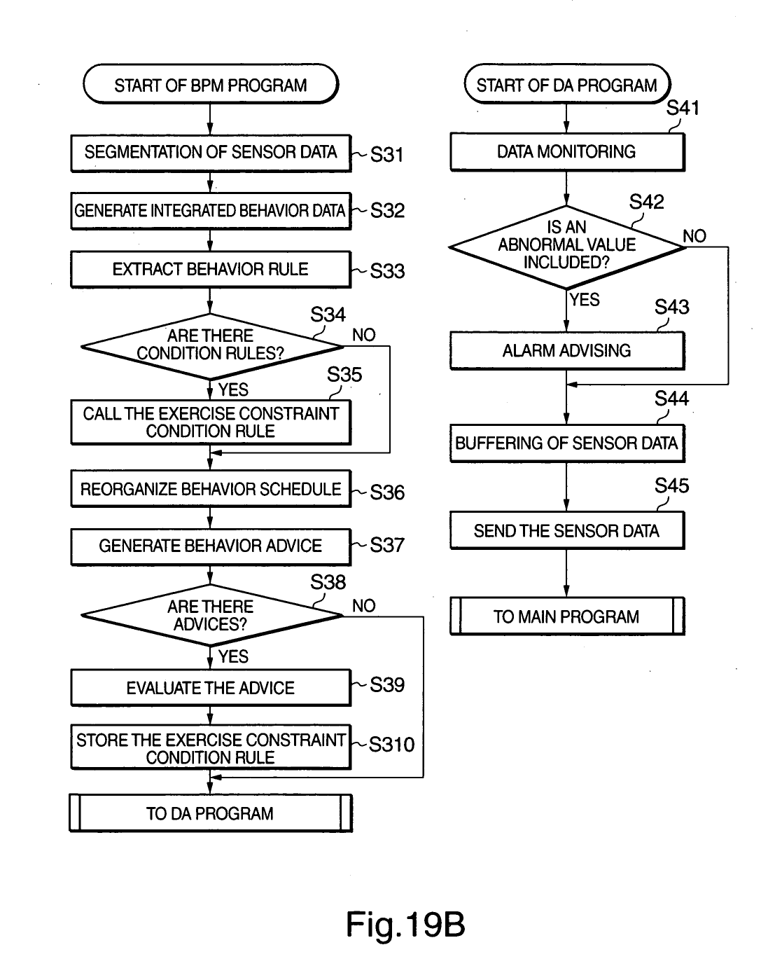

Next, operation of the above-mentioned components is explained using a daily control of the number of steps as an example. FIGS. 19A and 19B are a flow chart of one example of processing of the behavior control support apparatus. In this flow chart, eight phases “use preparation phase”, “set phase”, “monitoring phase”, “behavior description phase”, “behavior rule generation phase”, “scheduling phase”, “advising phase”, and “feedback phase” are explained.

<Use Preparation Phase>

First, before starting a main program, the main body unit C1, the sensor head C3, and the sensors C5 are fixed at a suitable place of the user's body by a belt or a clip. The sensor head C3 and the sensor group C5 are attached to a suitable place in accordance with a type of the sensor. In the case of using this apparatus as a pedometer, the sensor head C3 and the sensors C5 are preferably attached around the waist. In response to the user's switch-on of the main body unit C1, a program starts. First, a check whether the sensor operates normally and a calibration are executed. Then, the main program starts.

<Set Phase>

In FIG. 19A, if a user uses this program first (Yes at S11), personal attribute data of the user shown in FIG. 3 is input through a key pad of the main body unit C1 (S12). If the user used this program in the past (No at S11), the processing is forwarded to next log-in step by skipping the step S12. In the log-in step, by inputting a user name and a password, the system is activated as log-in (S13). Next, in the case of completing the system (Yes at S14), log-out is executed. In the case of not executing log-out (No at S14), the processing is forwarded to the next step. Next, if the behavior schedule data exists (Yes at S15), the behavior schedule data is input by reading from the behavior schedule data set 23, by describing through a scheduler or an editor, or by reading from another scheduler (S16). Then, the processing is forwarded to BDI (behavior data input) program. If the behavior schedule data does not exist (No at S15), the processing is forwarded to BDI program by skipping S16.

<Monitoring Phase>

In response to start of BDI program, if the behavior data is not input (No at S21), the processing is forwarded to DA (data analysis) program. In the DA program, sensor data is always sampled at a predetermined sampling rate, and the sampled value is monitored (S41). If the sensor data is above or below a threshold, or if the sensor data represents unusual pattern, the sensor data is regarded as unusual value (S43). In this case, the sensor data is stored as the sensor data set 3 while buffering.

<Behavior Description Phase>

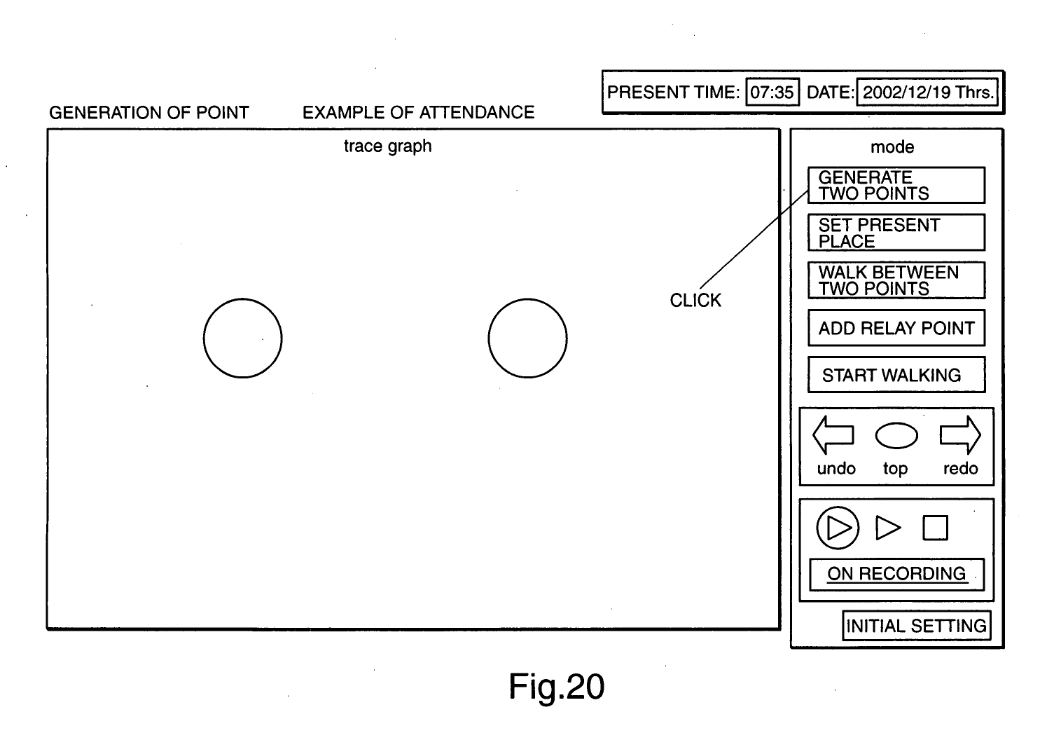

On the other hand, in the case of inputting behavior data (Yes at S21), first, the behavior data is input (S22). At S22, for example, in the case of inputting a behavior “attendance”, processes shown in FIGS. 20˜25 are presented.

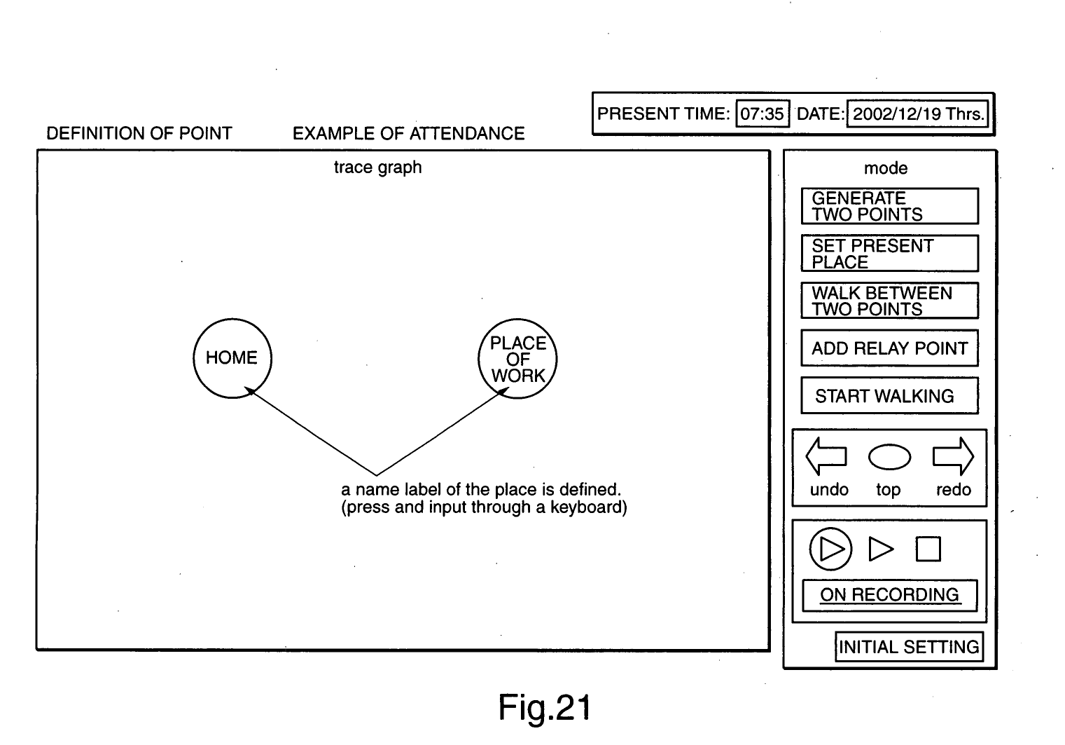

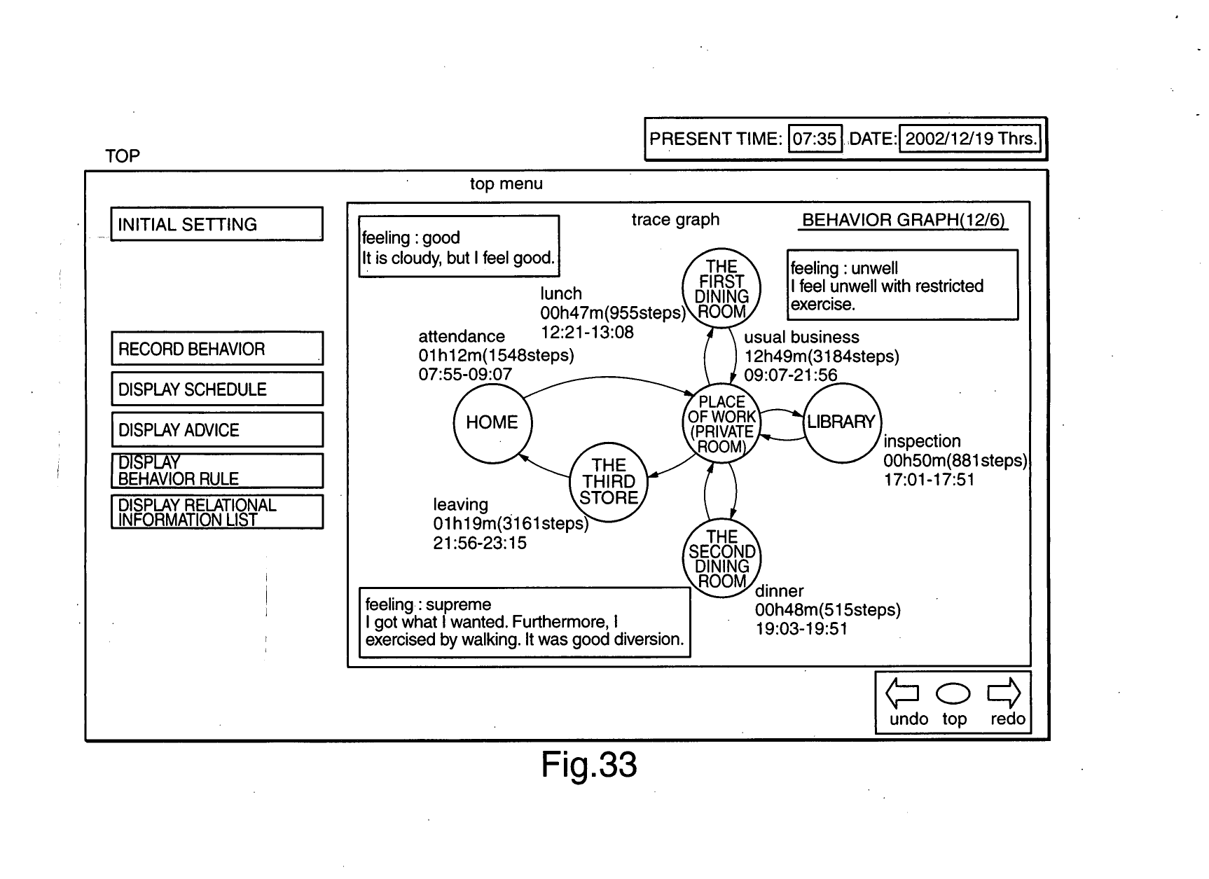

FIGS. 20˜25 are schematic diagrams of behavior description processing by using the data input interface C4 of the main body unit C1. First, when a mode button of “record of behavior” mode (shown in FIG. 33) of the data input interface C4 is clicked, a program status is forwarded to a behavior record mode. Next, when a button of generation of two points is clicked, a circular point node is presented on a display (FIG. 20). Next, as shown in FIG. 21, each point node is indicated by pressing, and a name of the place is defined using a keyboard. In FIG. 21, names of “home” and “place of work” are input. In this process, by preparing a selection item of names, the name may be selected using a drop down list.

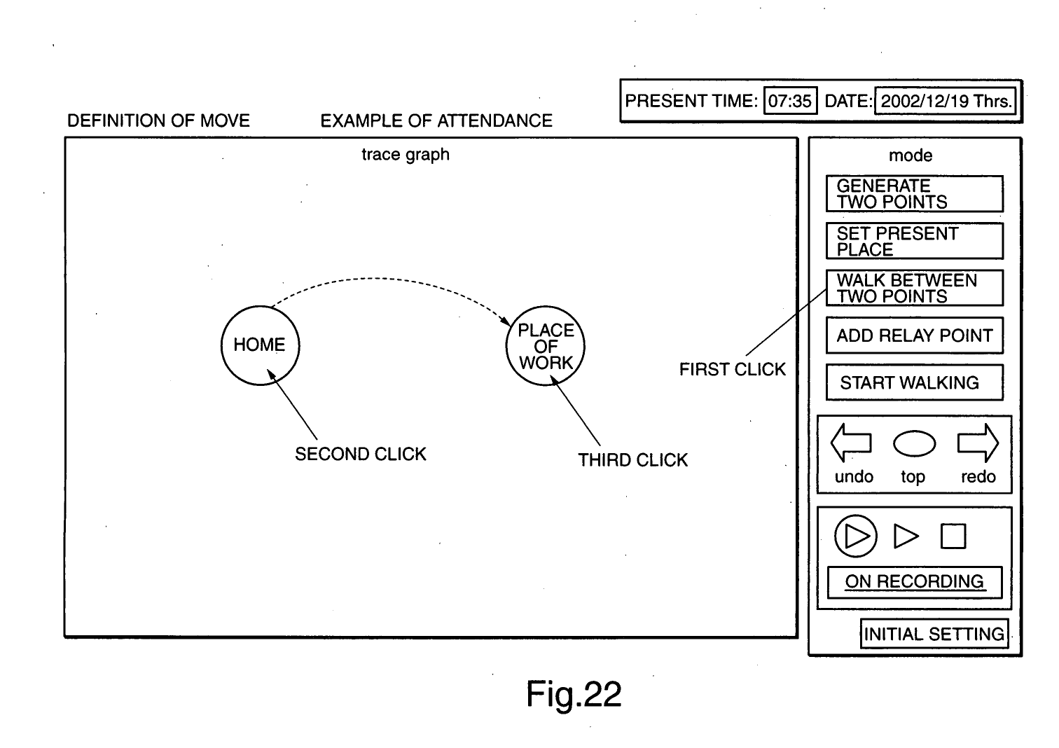

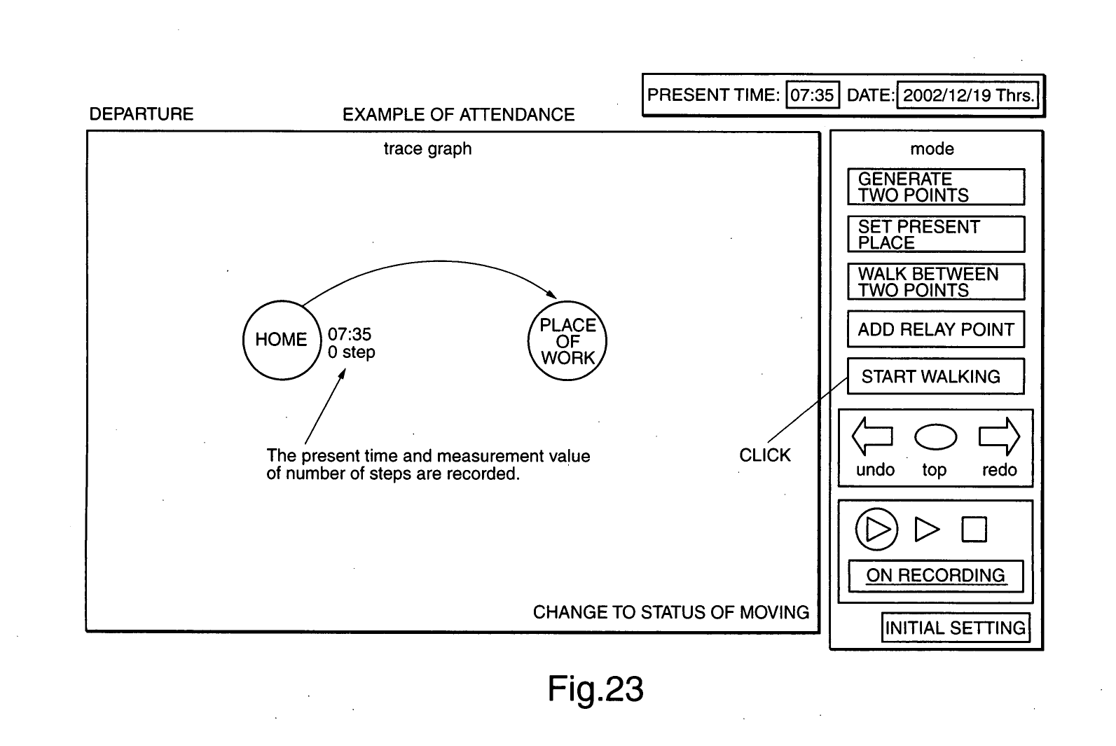

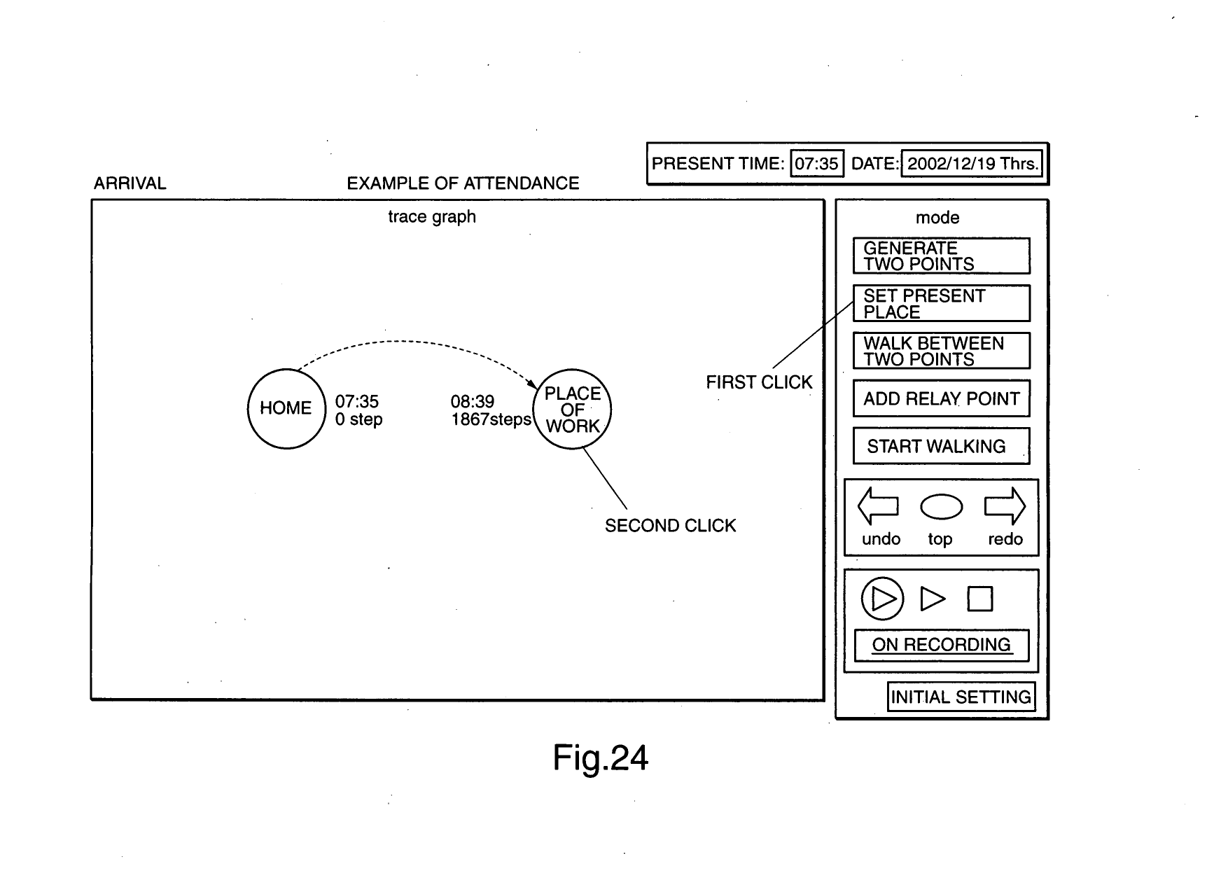

Next, as shown in FIG. 22, a button of move between two points is clicked at the time when the user begins to move. In this status, the present point node (home) is clicked first, and a destination point node (place of work) is clicked next. Then, as shown in FIG. 23, the present time and a measurement value of the number of steps are displayed near the present point node. This value is recorded. Furthermore, an arrow (arc) from the present point node to the destination node is displayed. In this status, if a button of start of move is clicked, the program is changed to a status of moving. When the user arrives at the destination, as shown in FIG. 24, the present place is changed by clicking a button of set of present place, and status of arrival to the destination is recorded.

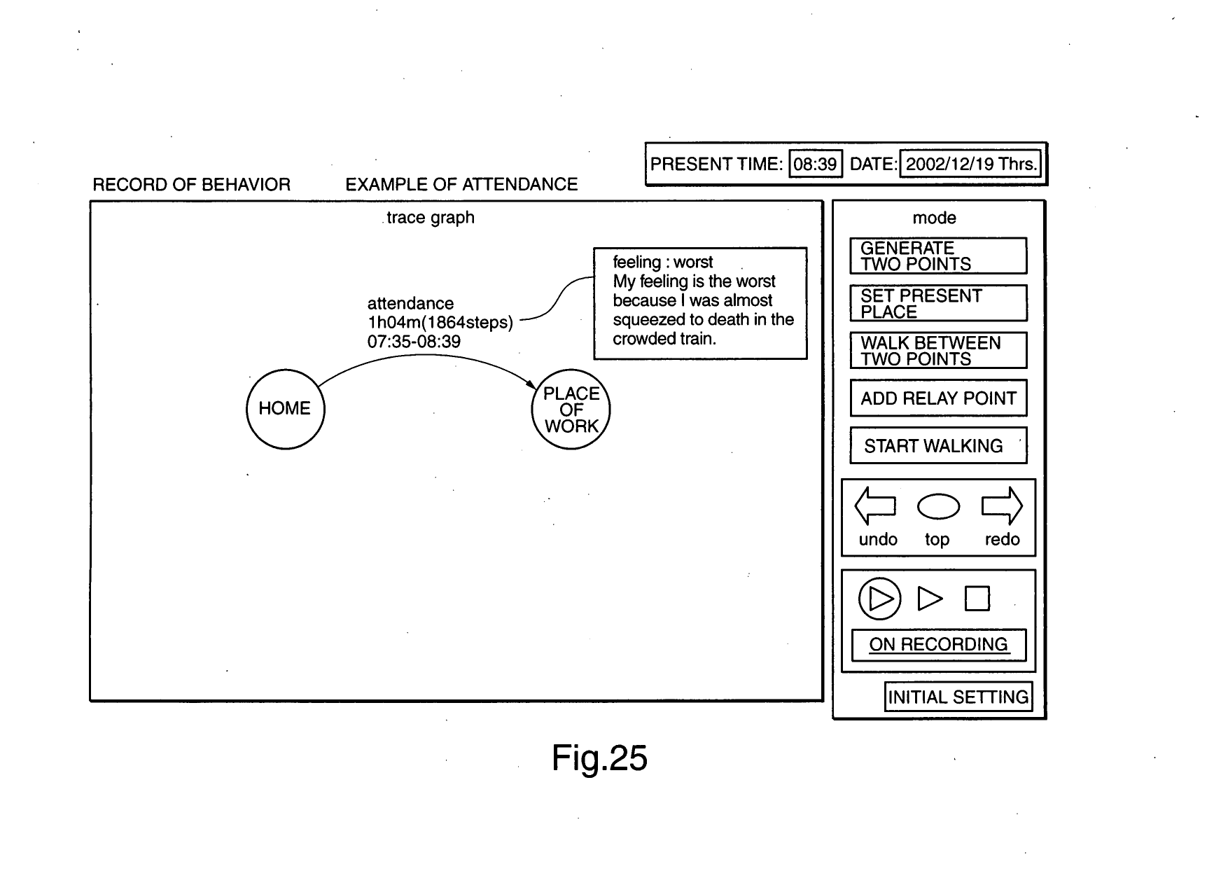

Then, the present arrival time and the measurement value of the number of steps are displayed near the destination point node (place of work), and these values are recorded. After several seconds from this status, a behavior summary described by above-mentioned steps is displayed on the arc, and this data is recorded. As shown in FIG. 25, the behavior summary including the behavior label, the moving time, the number of steps and the period is displayed on the arc between both points. The behavior label is defined as a pair of the departure point and the arrival point, and represents a name to specify the behavior. In FIG. 25, the behavior label is “attendance”. The behavior label is specified by referring to the behavior label set (FIG. 11). If a pair of the departure point and the arrival point is unknown, the user registers a new behavior label.

In the flow chart of FIG. 19A, if feeling data of each behavior is described (S23), a text box is displayed by clicking a predetermined button of the main body unit C1. The user's feeling is described in the text box, and the feeling data is input by linking this text box with a behavior abstract box (S24). By executing the above-mentioned basic steps, each behavior is added in order. The behavior data added are recorded in the apparatus.

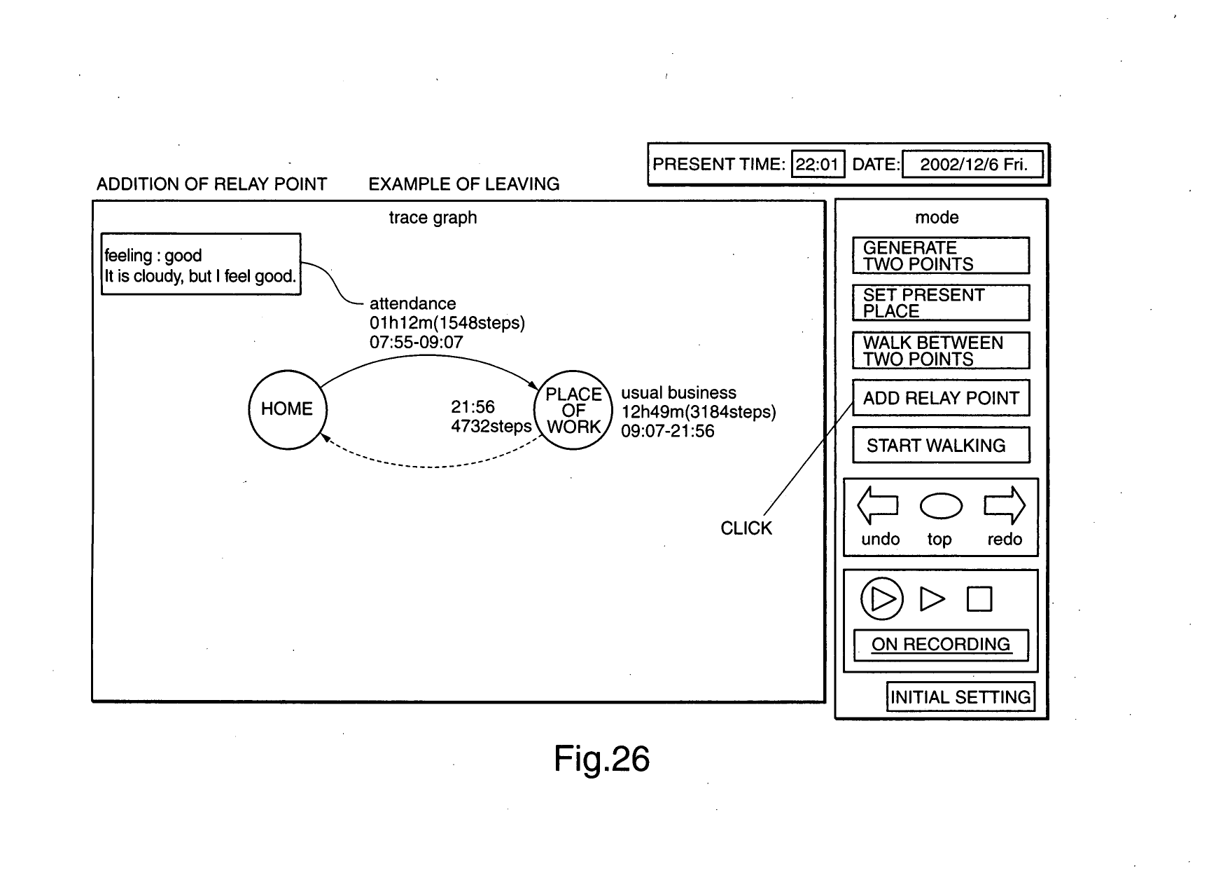

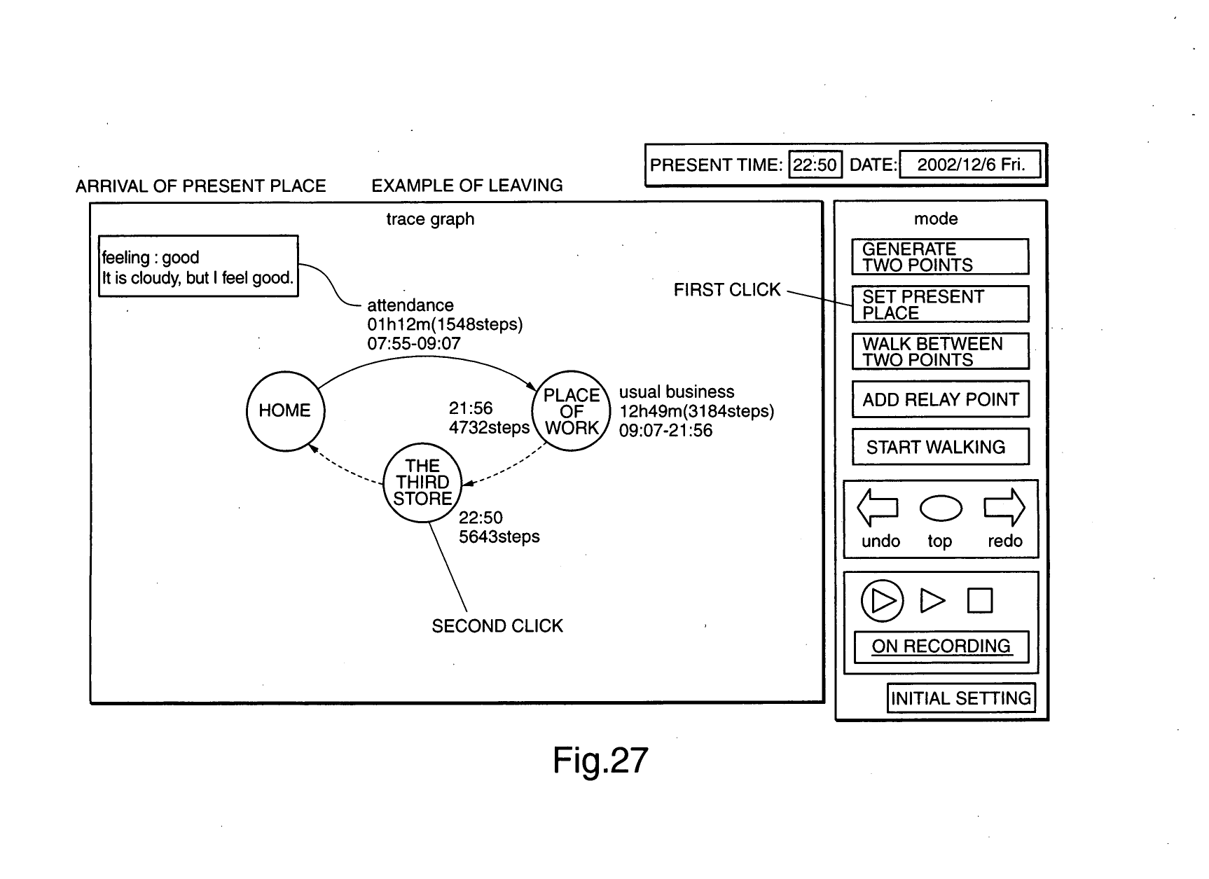

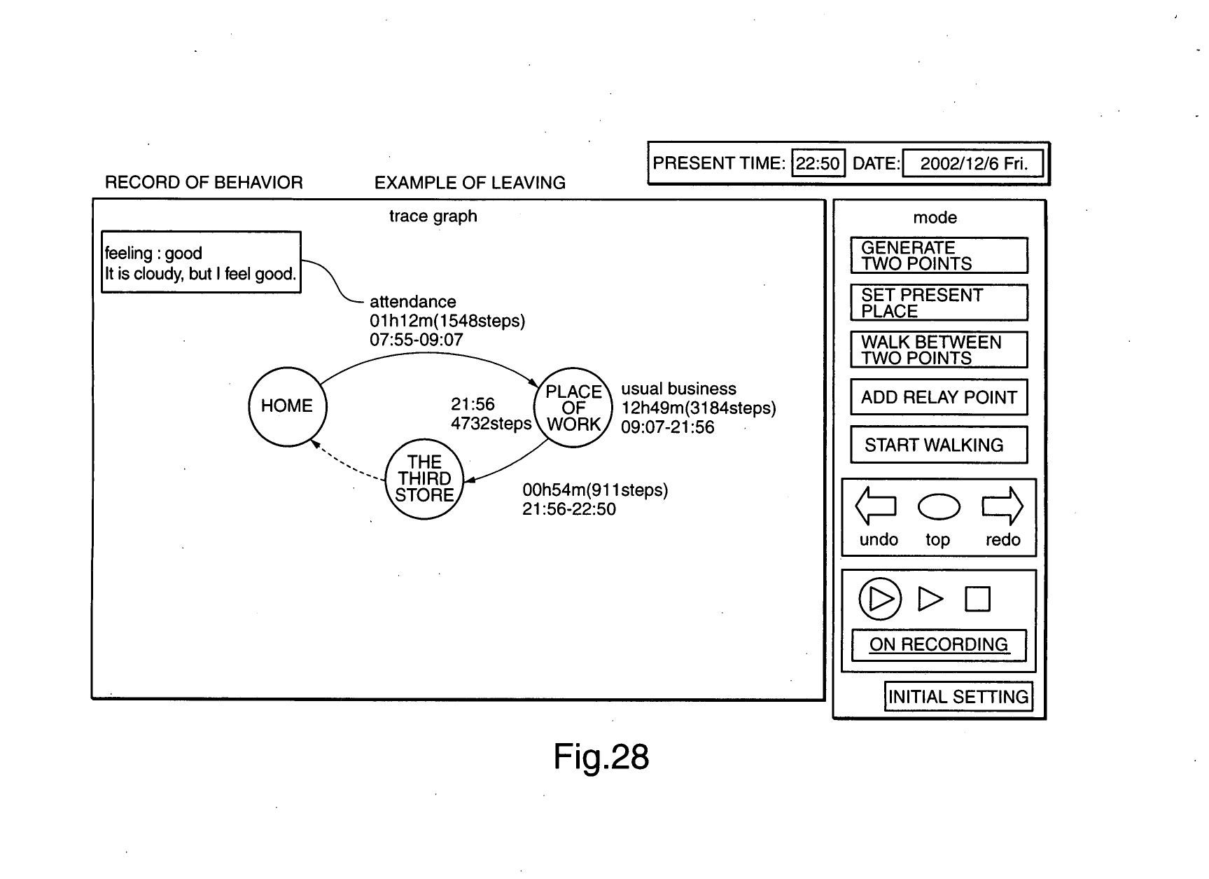

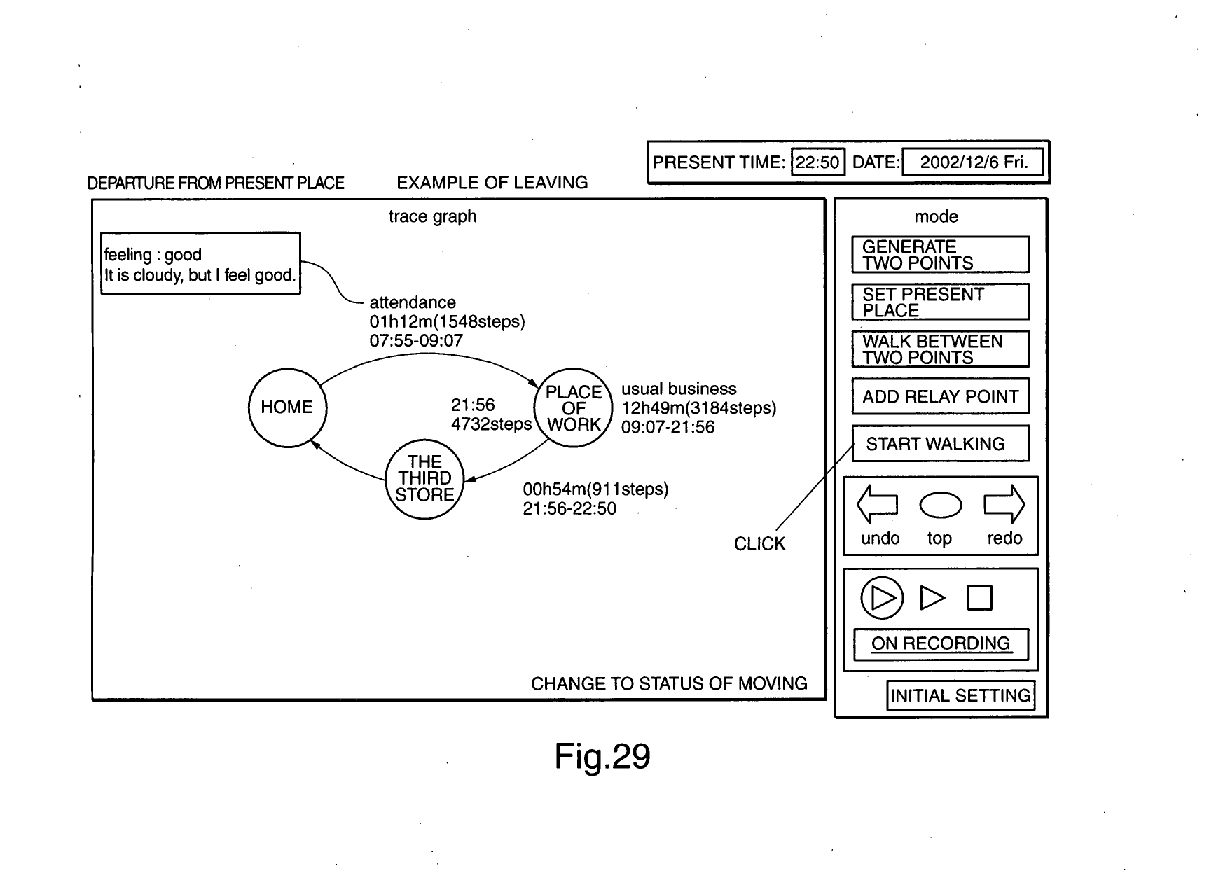

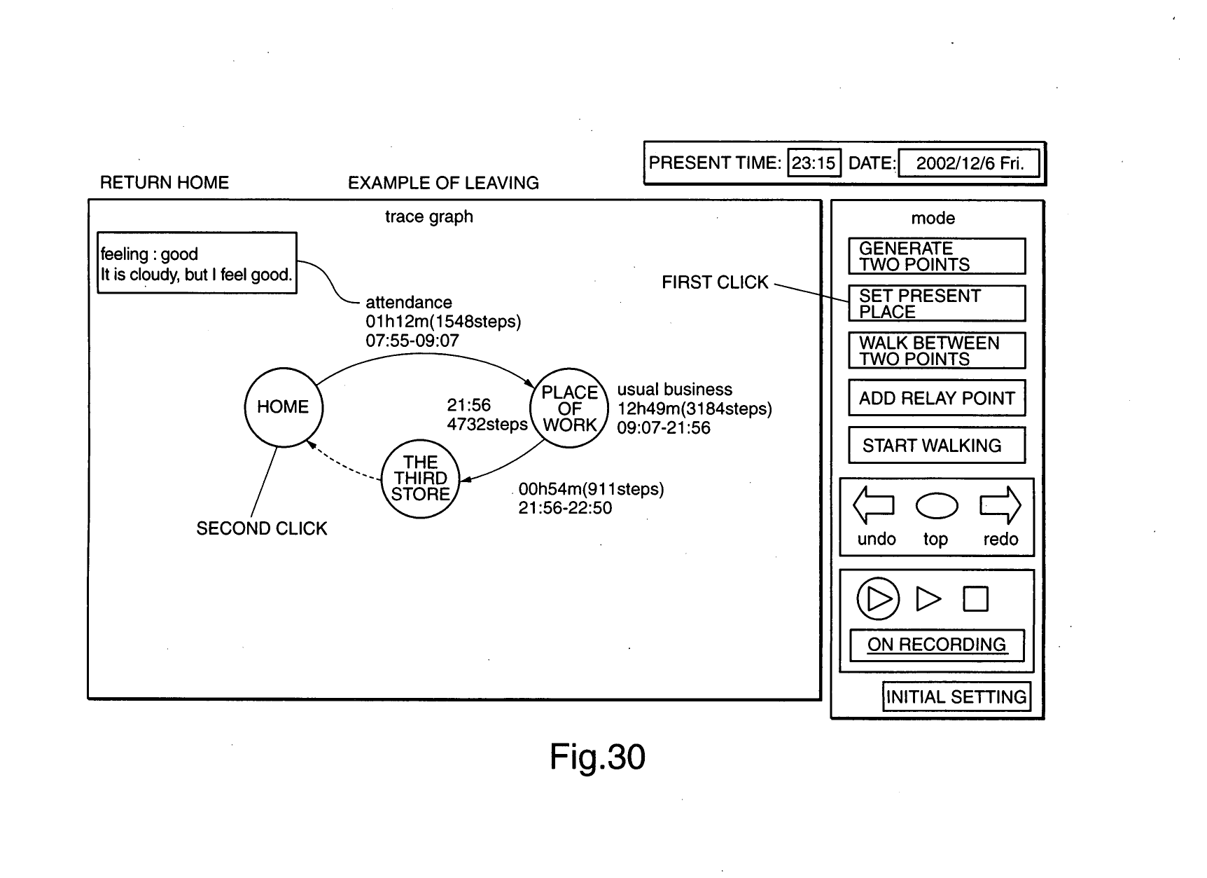

In the case of adding a behavior, for example, in the case that the user drops in at a place except for home on the way back from the office, a relay point is added first. Briefly, as shown in FIG. 26, a button of addition of relay point is clicked during the user's moving between two point nodes. Then, a new point node is displayed on the arc and a name of the place of the node is defined as shown in FIG. 26. When the user arrives at this relay point, a button of set of present place is clicked first, and a node of relay point is clicked second (FIG. 27). Then, as shown in FIG. 28, a behavior summary is generated in the same way with FIG. 25. Hereafter, a button of start of move is clicked (FIG. 29). When the user arrives at home, a button of set of present place is clicked (FIG. 30), and the behavior is described in the same way with above-mentioned processing. By executing these steps, in the main body unit C1, data as the behavior data set 21 (FIG. 4) and the feeling data 22 (FIG. 5) are stored.

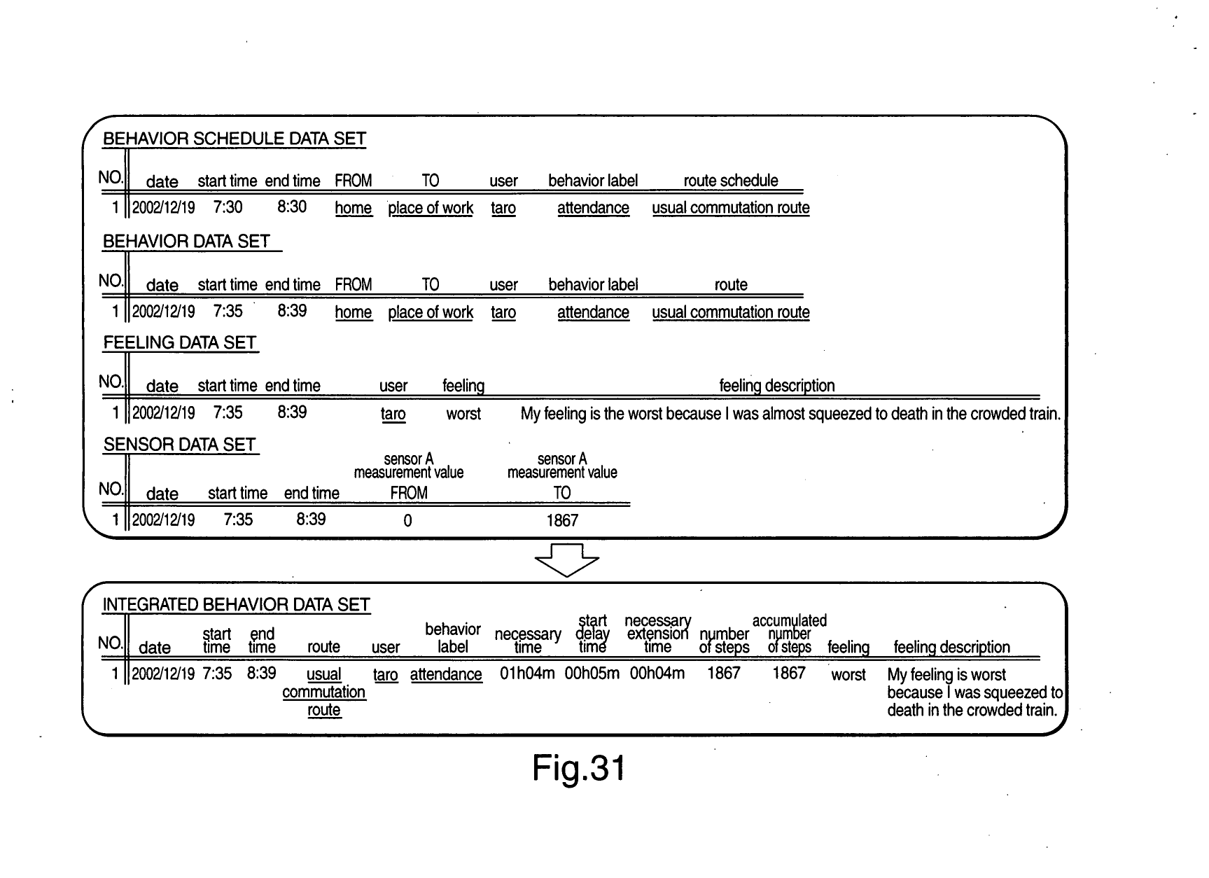

After S24 in the flow chart of FIG. 19A, the processing is forwarded to BPM (behavior process management) program. In this program, first, data stored in the sensor data set 3 is segmented based on the start time and the end time of the behavior data set 21 (S31). In this way, a biomedical status and a surrounding situation obtained from the sensor measurement value can be segmented for each behavior (segmentation). Next, the integrated behavior data generation unit 61 generates the integrated behavior data set 7 (FIG. 8) using the behavior data set 21, the feeling data set 22, the behavior schedule data set 23, the sensor data set 3, and the relational data set 9 (S32). FIG. 31 is a schematic diagram of process of generation of the integrated behavior data set 7. As shown in FIG. 31, the integrated behavior data set 7 is generated by integrally relating the behavior schedule data set 23, the behavior data set 21, the feeling data set 22, and the sensor data set 3. If necessary, data of the relational data set 9 is also referred.

<Behavior Rule Generation Phase>

Next, in FIG. 19B, the behavior rule generation unit 62 filters the integrated behavior data set 7 based on a predetermined basis, and generates the behavior rule set 9 (FIG. 9) by referring to the relational data set 9 (S33). In this case, for example, “behavior schedule constantly above a target value of number of steps”, “behavior changed to increase the number of steps”, and “behavior including good element as the feeling data” are used as the basis. Concretely, the behavior rule set 8 in FIG. 9 is generated from the integrated behavior data set 7 in FIG. 5. Furthermore, by using information obtained from the calendar weather data set (FIG. 12) at this phase, behavior difference based on weather of some day and behavior difference based on weekday are known.

<Scheduling Phase>

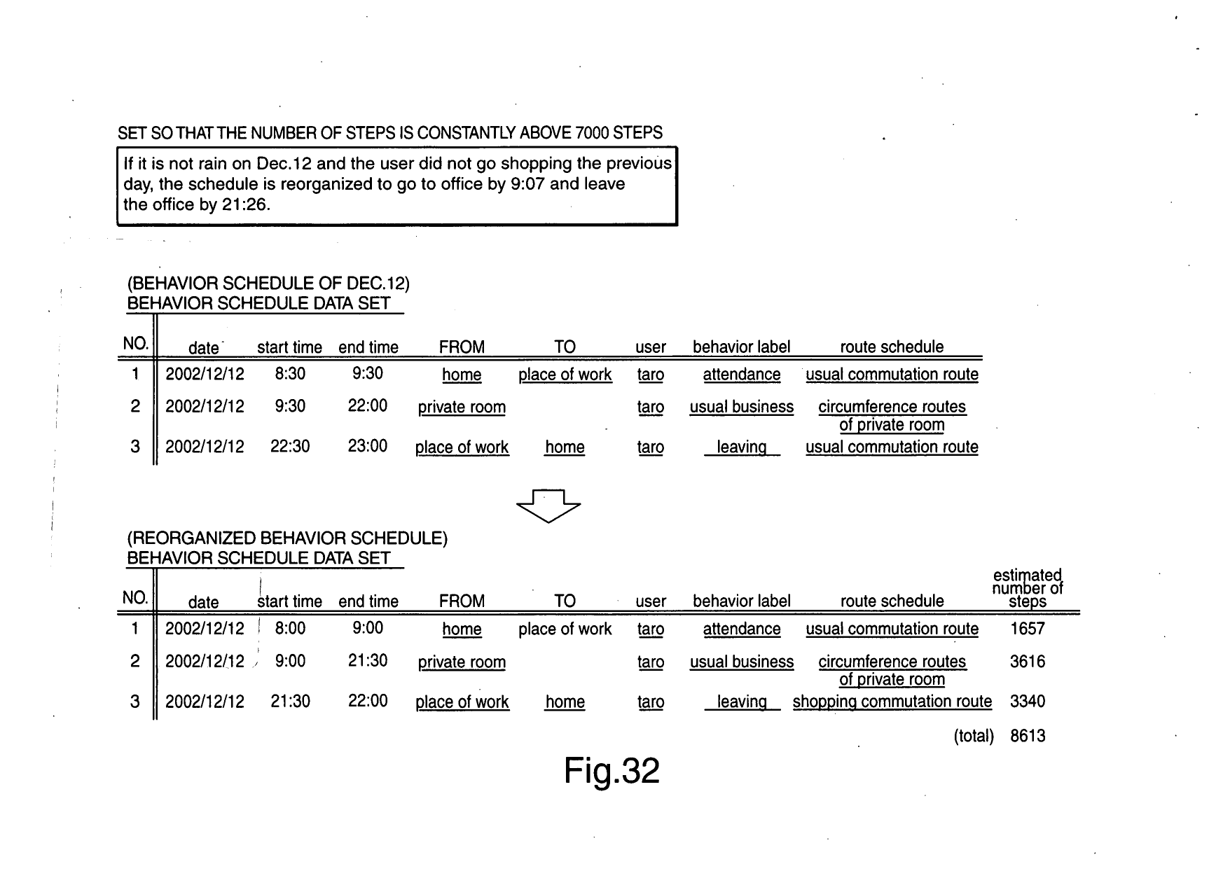

Next, the user's behavior is scheduled. First, it is decided whether the exercise constraint condition rule set 12 exists. If a condition rule does not exist (No at S34), the processing is forwarded to S36. If the condition rule exists (Yes at S34), the behavior schedule data set 23 is reorganized by referring to the exercise constraint condition rule set 12 (S36). FIG. 32 is a schematic diagram of process of organization of the behavior schedule data set 23. As shown in FIG. 32, the behavior schedule reorganization unit 63 adjusts the exercise quantity to urge the user to do efficient exercise by referring to the behavior rule set 8, and reorganizes the behavior schedule data set 23.

<Advising Phase>

Next, in FIG. 19B, the behavior advise generation unit 64 generates the behavior advice set 10 based on the behavior rule set 8 (S37). For example, this advice message is output through a display of the main body unit C1 in order to inform to the user. The advice message may be informed to the user by using speech or text mail.

<Feedback Phase>

Next, when the advice message is informed to the user (Yes at S38), the user evaluates the advice (S39). In this case, for example, four stages such as “A (It is good advice, and the user puts in practice.)”, “B (It is good advice, but the user does not put in practice.)”, “C (It is not good advice.)” and “D (It is an advice of wrong guess.)”, are selectively used.

By relating the advice evaluation with the behavior advice set 10, the exercise constraint condition rule set 12 is generated and stored in the database (S310). By using this exercise constraint condition rule set 12, the system can generate a soft advice matched with the user's behavior, liking and characteristics. Briefly, the advice evaluation input unit 65 integrates the user's evaluation for the advice with the behavior result, and stores the integrated result in the exercise constraint condition rule set 12. This condition rule is reused as input of the behavior schedule reorganization unit 63 and the behavior advice generation unit 64. These processing are repeated until the system is set as log-out (S14). Data generated by above-mentioned steps are displayed as a behavior graph. FIG. 33 is a schematic diagram of one example of the behavior graph. In this way, by using the present apparatus, the user's behavior of one day is visually arranged. Briefly, the present apparatus can be utilized as a self-control tool such as behavior control or office hours control.

As mentioned-above, in the present embodiment, the integrated behavior data generation unit 61 mutually relates the personal attribute data set 1, the behavior relational data group 2, the sensor data set 3, and the relational data set 9, and generates the integrated behavior data set 7. By referring to this integrated behavior data set 7, the behavior rule generation unit 62 generates the behavior rule set 8 comprising the user's behavior rule. Then, based on personal exercise's custom, characteristics, liking, and habit reflected on the behavior rule set 8, the advice to urge the user to exercise is presented to the user as a format representing an explicit reason. Accordingly, the system can generate a soft message matched with the user's behavior, liking, and characteristics. Furthermore, the exercise in daily life such as a walk or shopping can be inserted into a time segment. Accordingly, the behavior schedule reorganization unit 63 can reorganize the exercise plan at an interval in correspondence with change of the behavior schedule. Furthermore, even if the user is so busy that he can not do the exercise, by measuring the exercise quantity in daily life, the behavior advice generation unit 64 can promote an increase in the exercise quantity at familiar place.

Briefly, in the present embodiment, the user can know his/her exercise pattern and behavior rule, and naturally plan the exercise. By utilizing this specific feature, the user can naturally form a habit to do the exercise in daily life. As a result, this specific feature can be utilized as the user's health control.

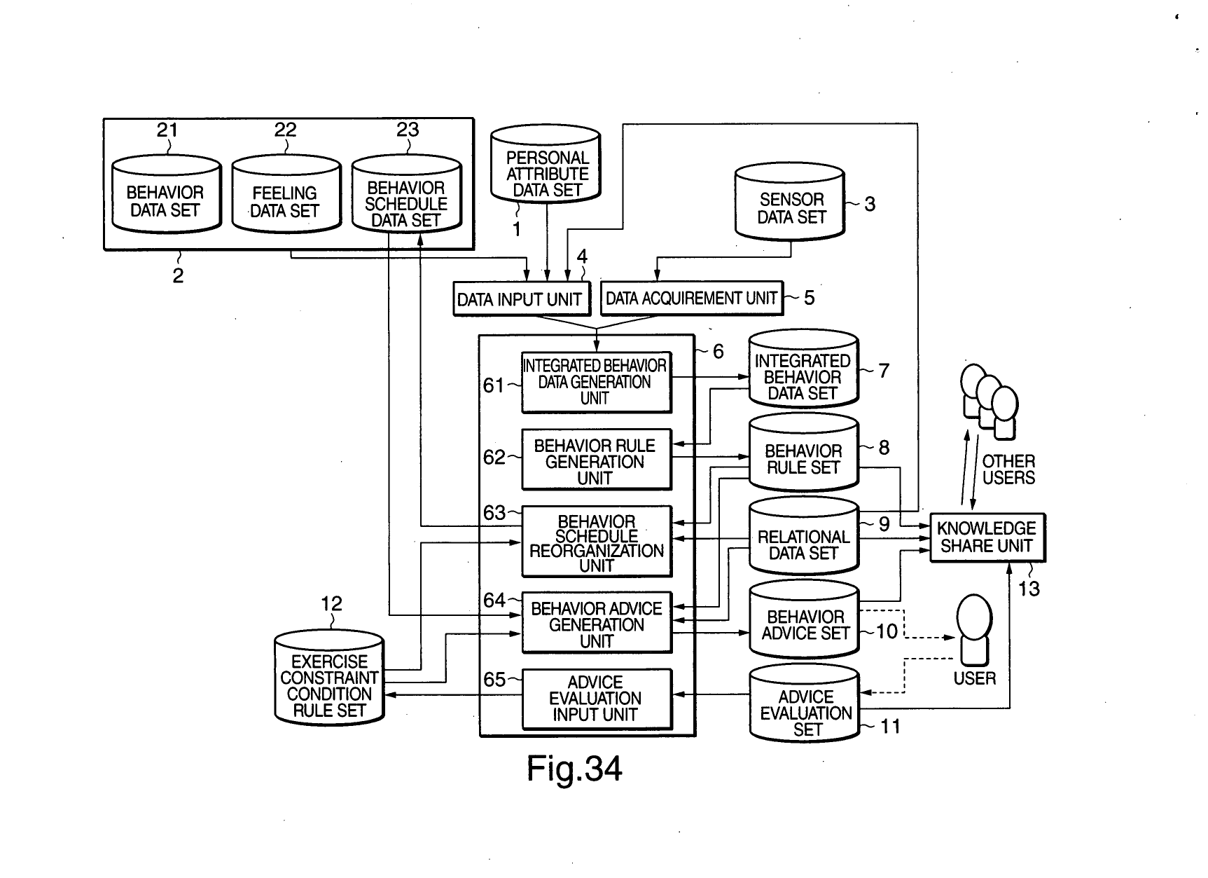

Next, a second embodiment is explained. FIG. 34 is a block diagram of the behavior control support apparatus of the second embodiment. As for a common unit (set) in FIGS. 2 and 34, the same number is assigned. A unit of FIG. 34 different from FIG. 2 is only explained. In addition to the components of FIG. 2, the system of FIG. 34 includes a knowledge share unit 13. The knowledge share unit 13 presents a base sharing the behavior rule set 8, the relational data set 9, the behavior advice set 10, the advice evaluation set 11, and the exercise constraint condition rule set 12 among a plurality of users. By sharing a database generated through the main body unit Cl possessed by each user, the knowledge and ability of controlling the exercise behavior can be shared. Accordingly, in the second embodiment, in addition to effect of the first embodiment, each user can mutually help in the case of being in trouble or being short of information.

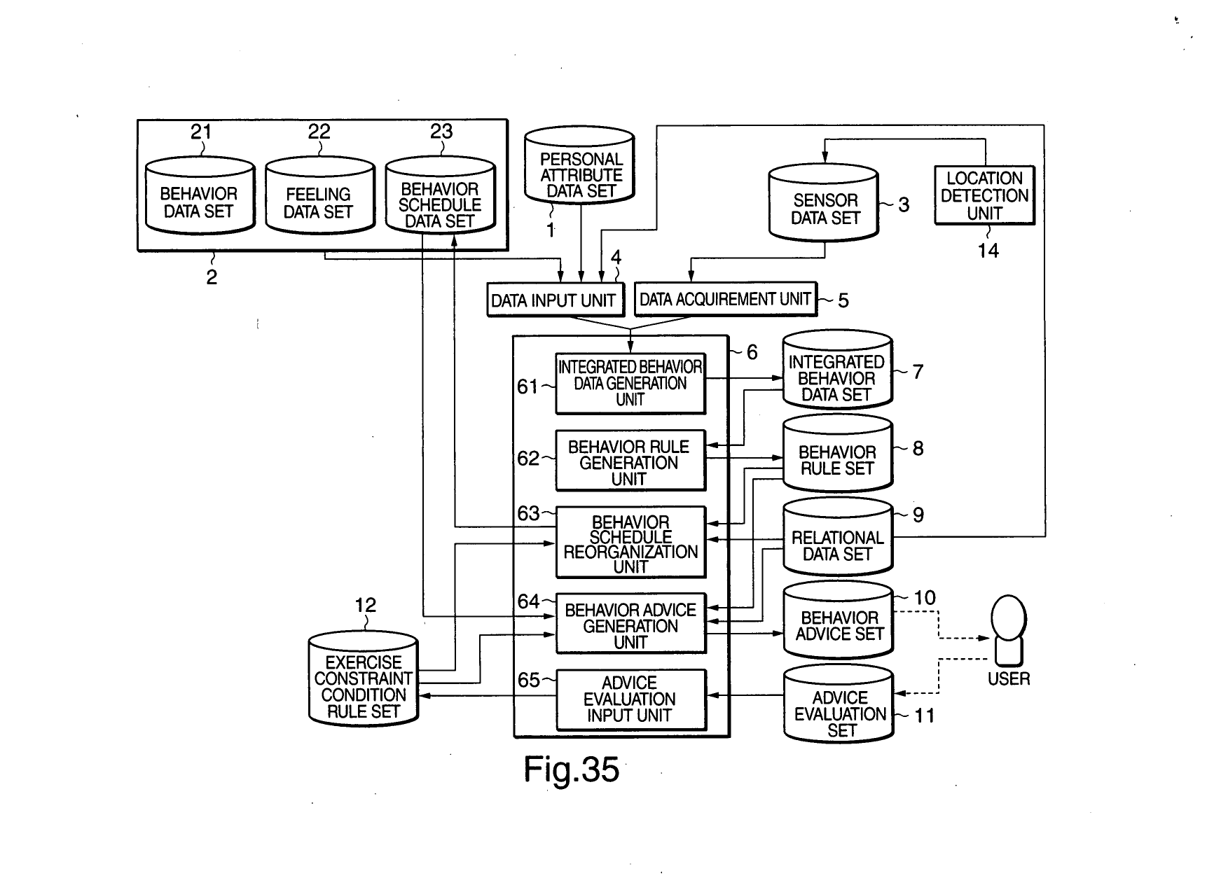

Next, a third embodiment is explained. FIG. 35 is a block diagram of the behavior control support apparatus of the third embodiment. As for a common unit (set) in FIGS. 2, 34, and 35, the same number is assigned. A unit of FIG. 35 different from FIGS. 2 and 34 is only explained. The system of FIG. 35 includes a location detection unit 14. The location detection unit 14 detects the user's location in time series and the user's location is recorded as one of the sensor data in the sensor data set 3. The location detection unit 14 is realized as a GPS (Global Positioning System) or an electronic check point set up on running courses, train stations, shops, hospitals, and so on, and a location specifying means such as a wireless tag (Radio Frequency Identification, RFID), an IC card or Bluetooth (registered trademark). In this component, by storing the location data obtained by the location detection unit 14 in the sensor data set 3, the user can omit his/her operation to input the present place. Accordingly, in the third embodiment, the user's burden can be further reduced, and the exercise behavior can be continually executed by raising his/her motivation.

As mentioned-above, in the present invention, the user can customize the exercise in daily life, and the user's exercise quantity can be naturally increased.

For embodiments of the present invention, the processing of the present invention can be accomplished by a computer-executable program, and this program can be realized in a computer-readable memory device.

In embodiments of the present invention, the memory device, such as a magnetic disk, a floppy disk, a hard disk, an optical disk (CD-ROM, CD-R, DVD, and so on), an optical magnetic disk (MD and so on) can be used to store instructions for causing a processor or a computer to perform the processes described above.

Furthermore, based on an indication of the program installed from the memory device to the computer, OS (operation system) operating on the computer, or MW (middle ware software), such as database management software or network, may execute one part of each processing to realize the embodiments.

Furthermore, the memory device is not limited to a device independent from the computer. By downloading a program transmitted through a LAN or the Internet, a memory device in which the program is stored is included. Furthermore, the memory device is not limited to one. In the case that the processing of the embodiments is executed by a plurality of memory devices, a plurality of memory devices may be included in the memory device. The component of the device may be arbitrarily composed.

In embodiments of the present invention, the computer executes each processing stage of the embodiments according to the program stored in the memory device. The computer may be one apparatus such as a personal computer or a system in which a plurality of processing apparatuses are connected through a network. Furthermore, in the present invention, the computer is not limited to a personal computer. Those skilled in the art will appreciate that a computer includes a processing unit in an information processor, a microcomputer, and so on. In short, the equipment and the apparatus that can execute the functions in embodiments of the present invention using the program are generally called the computer.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. An apparatus for supporting a user's behavior, comprising:
an integrated behavior database generation unit configured to generate an integrated behavior database correspondingly storing a biomedical information and a behavior relational information of the user, the biomedical information being detected by a sensor associated with the user's body;
a behavior rule generation unit configured to generate a behavior rule of the user by referring to the integrated behavior database;
a message generation unit configured to generate a message to urge the user to do an exercise by referring to the behavior rule; and
a message notice unit configured to notify the user of the message.

2. The apparatus according to claim 1,
wherein the behavior relational information comprises a behavior database, a feeling database, and a behavior schedule database.

3. The apparatus according to claim 2,
wherein the behavior database correspondingly includes a date, a start time, an end time, a start point, an end point, a user name, a behavior label, and a route.

4. The apparatus according to claim 3,
wherein the feeling database correspondingly includes a date, a start time, an end time, a user name, a feeling, and a feeling description.

5. The apparatus according to claim 4,
wherein the behavior schedule database correspondingly includes a date, a start time, an end time, a start point, an end point, a user name, a behavior label, and a route schedule.

6. The apparatus according to claim 5,
wherein the biomedical information comprises a sensor database, and
wherein the sensor database correspondingly includes a date, a start time, an end time, a measurement value of the sensor at the start time, and a measurement value of the sensor at the end time.

7. The apparatus according to claim 6,
wherein said integrated behavior data generation unit merges information of the behavior data set, the feeling data set and the behavior schedule data set for the same user, the same date, the same start time and the same end time, and generates the merged information as the integrated behavior database.

8. The apparatus according to claim 1,
wherein said behavior rule generation unit extracts a tendency of the user's behavior from information of the integrated behavior database, modifies the extracted information as a condition-result rule, and generates the condition-result rule as a behavior rule database.

9. The apparatus according to claim 1,
further comprising a relational database configured to store a conception dictionary data set, a behavior label set, a calendar weather data set, a route data set, a seat data set, a map data set, and a map relational data set, and
wherein said integrated behavior data generation unit adds information to the integrated behavior database by referring to each set of the relational database.

10. The apparatus according to claim 8,
further comprising a behavior schedule reorganization unit configured to reorganize information of the behavior schedule database by referring to the behavior rule database, and
wherein said message generation unit generates the message as an advice to urge the user to do the exercise by referring to the reorganized information of the behavior schedule database.

11. The apparatus according to claim 10,
further comprising a behavior advice database configured to store the message in correspondence with the behavior rule.

12. The apparatus according to claim 1, further comprising,
an advice evaluation input unit configured to input an evaluation for the message from the user, and
an advice evaluation database configured to store the evaluation in correspondence with the message.

13. The apparatus according to claim 12,
further comprising a constraint condition rule database configured to correspondingly store the behavior rule and the evaluation, and
wherein said message generation unit generates a message by referring to the constraint condition rule database.

14. The apparatus according to claim 5,
further comprising a data interface unit configured to input the feeling, the feeling description, and the behavior schedule data from the user.

15. The apparatus according to claim 14,
wherein said data interface unit interactively inputs a status data of the user's moving by the user's indication, and records the status data as the user's behavior in time series.

16. The apparatus according to claim 15,
wherein said data interface unit outputs a behavior graph of the user by using the recorded status data in time series.

17. The apparatus according to claim 13,
further comprising a database share unit configured to share information of the integrated behavior database and the constraint condition database among a plurality of users.

18. The apparatus according to claim 6,
further comprising a location detection unit configured to detect the user's location information, and
wherein the integrated behavior database correspondingly stores the biomedical information, the behavior relational information and the location information.

19. A method for supporting a user's behavior, comprising:
generating an integrated behavior database correspondingly storing a biomedical information and a behavior relational information of the user, the biomedical information being detected by a sensor associated with the user's body;
generating a behavior rule of the user by referring to the integrated behavior database;
generating a message to urge the user to do an exercise by referring to the behavior rule; and
notifying the user of the message.

20. A computer program product, comprising:
a computer readable program code embodied in said product for causing a computer to support a user's behavior, said computer readable program code comprising:
a first program code to generate an integrated behavior database correspondingly storing a biomedical information and a behavior relational information of the user, the biomedical information being detected by a sensor associated with the user's body;
a second program code to generate a behavior rule of the user by referring to the integrated behavior database;
a third program code to generate a message to urge the user to do an exercise by referring to the behavior rule; and
a fourth program code to notify the user of the message.
